SEQUENCE LISTING (50 sequences)

SEQ ID NO: 1 - protein, 6 aa, Synthetic construct
AAHFHK

SEQ ID NO: 2 - protein, 6 aa, Synthetic construct
KFHAWN

SEQ ID NO: 3 - protein, 6 aa, Synthetic construct
KHAWIF

SEQ ID NO: 4 - protein, 6 aa, Synthetic construct
NHNKIH

SEQ ID NO: 5 - protein, 6 aa, Synthetic construct
SHWHWA

SEQ ID NO: 6 - protein, 6 aa, Synthetic construct
KAKHKE

SEQ ID NO: 7 - protein, 6 aa, Synthetic construct
WNKKWK

SEQ ID NO: 8 - protein, 8 aa, Synthetic construct
ANAKIKKK

SEQ ID NO: 9 - protein, 8 aa, Synthetic construct
FEKWAKAH

SEQ ID NO: 10 - protein, 8 aa, Synthetic construct
FSHHSWKF

SEQ ID NO: 11 - protein, 8 aa, Synthetic construct
KAWFKHWN

SEQ ID NO: 12 - protein, 8 aa, Synthetic construct
KHSKAIAA

SEQ ID NO: 13 - protein, 8 aa, Synthetic construct
KWKHSHKW

SEQ ID NO: 14 - protein, 8 aa, Synthetic construct
WHAKKSHF

SEQ ID NO: 15 - protein, 8 aa, Synthetic construct
WHAKKSKF

SEQ ID NO: 16 - protein, 8 aa, Synthetic construct
AHIHFSEK

SEQ ID NO: 17 - protein, 8 aa, Synthetic construct
EHSSWHSK

SEQ ID NO: 18 - protein, 8 aa, Synthetic construct
IAKAKKWA

SEQ ID NO: 19 - protein, 8 aa, Synthetic construct
WNHSIFFH

SEQ ID NO: 20 - protein, 8 aa, Synthetic construct
NHSKKNIK

SEQ ID NO: 21 - protein, 8 aa, Synthetic construct
FKHNAISA

SEQ ID NO: 22 - protein, 8 aa, Synthetic construct
FHWNHWFH

SEQ ID NO: 23 - protein, 8 aa, Synthetic construct
ISSFEAAF

SEQ ID NO: 24 - protein, 9 aa, Synthetic construct
ANAKIKKKG

SEQ ID NO: 25 - protein, 9 aa, Synthetic construct
FEKWAKAHG

SEQ ID NO: 26 - protein, 9 aa, Synthetic construct
FSHHSWKFG

SEQ ID NO: 27 - protein, 9 aa, Synthetic construct
KAWFKHWNG

SEQ ID NO: 28 - protein, 9 aa, Synthetic construct
KHSKAIAAG

SEQ ID NO: 29 - protein, 9 aa, Synthetic construct
KWKHSHKWG

SEQ ID NO: 30 - protein, 9 aa, Synthetic construct
WHAKKSHFG

SEQ ID NO: 31 - protein, 9 aa, Synthetic construct
WHAKKSKFG

SEQ ID NO: 32 - protein, 7 aa, Synthetic construct
AAHFHKG

SEQ ID NO: 33 - protein, 7 aa, Synthetic construct
KHAWIFG

SEQ ID NO: 34 - protein, 7 aa, Synthetic construct
KFHAWNG

SEQ ID NO: 35 - protein, 7 aa, Synthetic construct
NHNKIHG

SEQ ID NO: 36 - protein, 7 aa, Synthetic construct
SHWHWAG

SEQ ID NO: 37 - protein, 9 aa, Synthetic construct
AAHFHKGSG

SEQ ID NO: 38 - protein, 11 aa, Synthetic construct
ANAKIKKKGSG

SEQ ID NO: 39 - protein, 11 aa, Synthetic construct
FEKWAKAHGSG

SEQ ID NO: 40 - protein, 11 aa, Synthetic construct
FSHHSWKFGSG

SEQ ID NO: 41 - protein, 11 aa, Synthetic construct
KAWFKHWNGSG

SEQ ID NO: 42 - protein, 11 aa, Synthetic construct
KHSKAIAAGSG

SEQ ID NO: 43 - protein, 11 aa, Synthetic construct
KWKHSHKWGSG

SEQ ID NO: 44 - protein, 11 aa, Synthetic construct
WFNHKSKGGSG

SEQ ID NO: 45 - protein, 11 aa, Synthetic construct
WHAKKSHFGSG

SEQ ID NO: 46 - protein, 11 aa, Synthetic construct
WHAKKSKFGSG

SEQ ID NO: 47 - protein, 9 aa, Synthetic construct
KFHAWNGSG

SEQ ID NO: 48 - protein, 9 aa, Synthetic construct
KHAWIFGSG

SEQ ID NO: 49 - protein, 9 aa, Synthetic construct
NHNKIHGSG

SEQ ID NO: 50 - protein, 9 aa, Synthetic construct
SHWHWAGSG

(19) United States
(12) Patent Application Publication
(10) Pub. No.: US 2024/0174712 A1
(43) Pub. Date: May 30, 2024

(54) COMPOSITIONS AND METHODS FOR PURIFYING ALPHA-1 ANTITRYPSIN USING PEPTIDE LIGANDS

(71) Applicant: North Carolina State University, Raleigh, NC (US)

(72) Inventors: Stefano Menegatti, Raleigh, NC (US); Wenning Chu, Raleigh, NC (US)

(21) Appl. No.: 18/060,215

(22) Filed: Nov. 30, 2022

(51) Int. Cl. C07K 1/32

(52) U.S. Cl. CPC C07K 1/32

(57) ABSTRACT

The present disclosure provides compositions and methods related to the purification and/or isolation of α-1 antitrypsin (AAT). In particular, the present disclosure provides novel peptide ligands capable of binding recombinantly produced α-1 antitrypsin to facilitate its isolation and/or purification from processing fluid streams for subsequent therapeutic use.

### STATEMENT REGARDING FEDERALLY SPONSORED RESEARCH OR DEVELOPMENT

This invention was made with government support under grant number CBET1653590 awarded by the National Science Foundation. The government has certain rights in the invention.

### SEQUENCE LISTING STATEMENT

The contents of the electronic sequence listing titled (41350_151_SequenceListing.xml; Size: 43,997 bytes; and Date of Creation: May 17, 2023) is herein incorporated by reference in its entirety.

### FIELD

The present disclosure provides compositions and methods related to the purification and/or isolation of α-1 antitrypsin (AAT). In particular, the present disclosure provides novel peptide ligands capable of binding recombinantly produced α-1 antitrypsin to facilitate its isolation and/or purification from processing fluid streams for subsequent therapeutic use.

### BACKGROUND

Recent α-1 antitrypsin (AAT) deficiency is an inherited disorder that frequently causes chronic obstructive pulmonary (COPD) and liver diseases, and—more rarely—inflammation of the skin (panniculitis) or blood vessels (vasculitis). AAT deficiency occurs worldwide, although its prevalence varies by population, and affects about 1 in 1,500 to 3,500 individuals with European ancestry. Augmentation therapy, otherwise called replacement therapy, is a treatment available to patients with AAT deficiency and aims to increase the blood level of AAT by supplementing the purified human protein by intravenous infusion.

Current AAT manufacturing processes utilize human plasma as a source, owing to its wide availability, in particular its Cohn Fraction IV precipitate: the cleavage of disulfide bonds of all proteins in Fraction IV using a reducing reagent, typically dithiothreitol, followed the centrifugation of the resulting precipitate affords an AAT-enriched solution that is suitable for chromatographic purification. However, the complexity of plasma fractionation, the loss of proteins during AAT enrichment from Cohn fraction IV, the need of subsequent chromatographic processing, and the risk of transmitting infectious agents from pooled plasma to the AATD patients limit the attractiveness of this process.

The recent advancements in engineering cell lines capable of protein expression and secretion at high titer point towards recombinant production as a viable alternative to meet the clinical demand of human AAT. This places the focus on the downstream pipeline and the availability of robust chromatographic adsorbents enabling affordable purification at scale. Currently, Alpha-1 Antitrypsin Select—henceforth, AAT Select—is the only affinity resin commercially available for AAT purification. By relying on a camelid antibody fragment for AAT capture, this resin features high binding capacity (˜10 mg of AAT per mL of resin) and selectivity; however, it also presents the characteristic limitations of affinity absorbents functionalized with protein ligands, namely high cost and lability towards the proteases in the feedstocks and caustic cleaning-in-place, which can cause the release of immunogenic fragments in the product stream and shorten the resin's lifetime.

### SUMMARY

Embodiments of the present disclosure include novel peptide ligands capable of binding α-1 antitrypsin (AAT). In some embodiments, the peptide ligand comprises at least 6 amino acids and (i) at least one histidine residue and/or at least one lysine residue; (ii) at least one asparagine residue, and/or at least one serine residue, and/or at least one glutamate residue; or (iii) at least one phenylalanine residue and/or at least one tryptophan residue; and (iv) the peptide ligand binds α-1 antitrypsin. In some embodiments, the at least one peptide ligand further comprises an alanine residue.

In some embodiments, the peptide ligand binds human α-1 antitrypsin at a binding site comprising at least one of the following characteristics: (i) a pocket depth from about 12.5 Å to about 14.5 Å; (ii) a volume from about 1100 Å³ to about 1800 Å³; (iii) a pI from about 3.5 to about 5.5; and/or (iv) a GRAVY index from about −0.05 to about −0.65.

In some embodiments, the peptide ligand exhibits a maximum equilibrium binding capacity (Qₘₐₓ) of at least about 3.0 mg of α-1 antitrypsin per mL resin. In some embodiments, the peptide ligand exhibits a disassociation constant (K_{D}) less than or equal to about 7.0×10⁻⁶ M. In some embodiments, the peptide ligand exhibits a dynamic binding capacity (DBC₁₀%) from about 5 mg/mL to about 20 mg/mL.

In some embodiments, the peptide ligand comprises an amino acid sequence having at least 90% sequence identity with any one of SEQ ID NOs: 1-23.

In some embodiments, the peptide ligand comprises at least 6 amino acids and an amino acid sequence having at least 90% sequence identity with any one of SEQ ID NOs: 1-7. In some embodiments, the peptide ligand comprises the amino acid sequence of any one of SEQ ID NOs: 1-5. In some embodiments, the peptide ligand comprises the amino acid sequence of any one of SEQ ID NOs: 6 or 7.

In some embodiments, the peptide ligand comprises at least 8 amino acids and an amino acid sequence having at least 90% sequence identity with any one of SEQ ID NOs: 8-23. In some embodiments, the peptide ligand comprises the amino acid sequence of any one of SEQ ID NOs: 8-15. In some embodiments, the peptide ligand comprises the amino acid sequence of any one of SEQ ID NOs: 16-23.

In some embodiments, the peptide ligand comprises a linker, and wherein the linker is capable of binding to a solid support. In some embodiments, the linker is bound to the C-terminus of the peptide ligand, and wherein the linker comprises a Glyₙ or a [Gly-Ser-Gly]m, wherein 10≥n≥1 and 6≥m≥1.

In some embodiments, the peptide ligand is bound to a solid support. In some embodiments, the solid support comprises a non-porous or porous particle, a membrane, a polymer surface, a fiber or a woven or non-woven fibermat, a hydrogel, a microplate, and/or a microfluidic device. In some embodiments, the solid support comprises polyacrylate, polyacrylamide, polyether, polyolefin, polyester, polysaccharide, iron oxide, silica, titania, and/or zirconia.

Embodiments of the present disclosure also include a composition for purifying α-1 antitrypsin, or a fragment thereof, from a fluid, wherein the composition comprises any of the peptide ligands described herein.

In some embodiments, the fluid is a cell culture fluid. In some embodiments, the fluid comprises a supernatant and/or a cellular lysate. In some embodiments, the fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof. In some embodiments, the fluid is derived from HEK293 cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the fluid comprises a pH from about 6.0 to about 9.0. In some embodiments, the fluid is derived from yeast cells or fungal cells. In some embodiments, the yeast cells are Pichia pastoris, Saccharomyces cerevisiae, or Saccharomyces boulardii cells.

Embodiments of the present disclosure also include an adsorbent for purifying α-1 antitrypsin from a fluid, wherein the adsorbent comprises any of the peptide ligands described herein.

Embodiments of the present disclosure also include a method of purifying α-1 antitrypsin, or a fragment thereof, from a fluid using any of the peptide ligands described herein. In some embodiments, the method includes contacting the fluid with any of the peptide ligands described herein, or any of the adsorbents described herein, under conditions sufficient for the peptide ligands to bind the α-1 antitrypsin; and eluting the α-1 antitrypsin from the peptide ligands.

In some embodiments, the conditions sufficient for the peptide ligands to bind the α-1 antitrypsin comprise use of a buffer comprising: (i) 0.1-0.2 M NaCl in 10⁻³⁰ mM Bis-Tris HCl buffer at pH 5.5 to 6.5; (ii) 0.1-0.2 M NaCl in 40-60 mM Tris HCl buffer at pH 7.0 to pH 8.0; or (iii) PBS buffer at pH 7.0 to pH 8.0.

In some embodiments, eluting the α-1 antitrypsin from the peptide ligands comprises use of a buffer comprising 1-3 M MgCl₂ in PBS at pH 7.0 to 8.0.

In some embodiments, the method results in a yield for the α-1 antitrypsin of at least 50%. In some embodiments, the method results in a purity for the α-1 antitrypsin of at least 89%.

### BRIEF DESCRIPTION OF THE DRAWINGS

FIGS. 1A-1B: Identification of AAT-binding peptides. (FIG. 1A) A library of peptide-ChemMatrix beads was (1) incubated with a screening feed comprising AF594-labeled AAT and AF488-labeled CHO host cell proteins, washed, and (2) fed to the bead-sorting microfluidic device; (3) green-only and red-and-green beads were discarded, whereas the red-only beads were selected as positive; (4) the selected beads were regenerated and re-entered into the screening loop; (5) finally, the positive beads were analyzed by Edman degradation to identify candidate AAT-binding sequences. (FIG. 1B) Sequence homology of selected 8-mer and 6-mer peptides.

FIG. 2: Adsorption isotherms of AAT on selected peptide-Toyopearl adsorbents and reference AAT Select resin.

FIGS. 3A-3B: (FIG. 3A) front and (FIG. 3B) back view of the representative complexes formed by peptides KAWFKHWNGSG (yellow cartoon; SEQ ID NO: 27), WHAKKSHFGSG (blue cartoon; SEQ ID NO: 30), KWKHSHKWGSG (red cartoon; SEQ ID NO: 29), and WHAKKSKFGSG (green cartoon; SEQ ID NO: 31) with human AAT (PDB ID: 1HP7, 5IO1, 4PYW; grey cartoon) obtained via molecular docking and dynamics simulations; the targeted binding sites are red, blue, green, and orange mesh.

FIGS. 4A-4D: Values of dynamic binding capacity of 10% AAT flow-through (DBC₁₀%, mg of AAT per mL resin) measured at two values of residence time (RT: 2 and 5 min) and four different buffer compositions and pHs, namely (FIG. 4A) PBS at 7.4, (FIG. 4B) 50 mM Tris-HCl at 7.4, (FIG. 4C) 20 mM Bis-Tris-HCl at 6.0, and (FIG. 4D) 50 mM Tris-HCl at pH 8.0); Resin 1: Alpha-1 antitrypsin Select; Resin 2: WHAKKSKFG-Toyopearl (SEQ ID NO: 31); Resin 3: WHAKKSHFG-Toyopearl (SEQ ID NO: 30); Resin 4: KWKHSHKWG-Toyopearl (SEQ ID NO: 29); Resin 5: FSHHSWKFG-Toyopearl (SEQ ID NO: 26); Resin 6: KAWFKHWNG-Toyopearl (SEQ ID NO: 27); Resin 7: AAHFHKG-Toyopearl (SEQ ID NO: 32); Resin 8: KENHWNG-Toyopearl; Resin 9: KHSKAIAAG-Toyopearl (SEQ ID NO: 28); Resin 10: KHAWIFG-Toyopearl (SEQ ID NO: 33); Resin 11: SHWHWAG-Toyopearl (SEQ ID NO: 36); Resin 12: NHNKIHG-Toyopearl (SEQ ID NO: 35); Resin 13: FEKWAKAHG-Toyopearl (SEQ ID NO: 25); Resin 14: ANAKIKKKG-Toyopearl (SEQ ID NO: 24).

FIGS. 5A-5B: (FIG. 5A) SDS-PAGE analysis (reducing condition, silver staining) of the chromatographic fractions obtained by purifying AAT from CHO cell culture fluids using AAT Select, WHAKKSKFG-Toyopearl (SEQ ID NO: 31) and WHAKKSHFG-Toyopearl (SEQ ID NO: 30) resins. Labels: MW, molecular weight marker; AAT, alpha-1 antitrypsin standard; Feed, feedstock (AAT titer: 1.15 mg/mL; HCP titer: 0.5 mg/mL); FT, flow-through fraction; E, eluted fraction in 2M MgCl₂ in PBS at pH 7.4; R, regeneration fraction in 0.1 M glycine at pH 2.5. (FIG. 5B) SEC analysis of pure AAT, AAT spiked in CHO-S CCF, and AAT purified using AAT Select, WHAKKSKFG-Toyopearl (SEQ ID NO: 31) and WHAKKSHFG-Toyopearl (SEQ ID NO: 30) resins.

FIG. 6: Lifetime study of WHAKKSHFG-Toyopearl (SEQ ID NO: 30) vs. AAT Select resins: values of AAT yield normalized against the AAT yield of the fresh resin measured throughout subsequent cycles of AAT purification, and resin regeneration and alkaline sanitization.

FIGS. 7A-7B: Druggability study of human AAT. (FIG. 7A) Structure of human AAT (PDB ID: 1HP7, 5IO1, 4PYW), comprising nine α-helices and three β-pleated sheets; (FIG. 7B) putative binding site 1 (red), site 2 (green), site 3 (blue), site 4 (yellow), and site 5 (pink) identified on the AAT core structure (grey cartoon) using SiteMap.

FIGS. 8A-8B: Complexes formed by peptides (FIG. 8A) AAHFHK-GSG (SEQ ID NO: 37), (FIG. 8B) ANAKIKKK-GSG (SEQ ID NO: 38), (FIG. 8C) FEKWAKAH-GSG (SEQ ID NO: 39), (FIG. 8D) FSHHSWKF-GSG (SEQ ID NO: 40), (FIG. 8E) KAWFKHWN-GSG (SEQ ID NO: 41), (FIG. 8F) KFHAWN-GSG (SEQ ID NO: 47), (FIG. 8G) KHAWIF-GSG (SEQ ID NO: 48), (FIG. 8H) KHSKAIAA-GSG (SEQ ID NO: 42), (FIG. 8I) KWKHSHKW-GSG (SEQ ID NO: 43), (FIG. 8J) NHNKIH-GSG (SEQ ID NO: 49), (FIG. 8K) SHWHWA-GSG (SEQ ID NO: 50), (FIG. 8L) WHAKKSHF-GSG (SEQ ID NO: 45), (FIG. 8M) WHAKKSKF-GSG (SEQ ID NO: 46), and (FIG. 8N) WFNHKSKG-GSG with human AAT (PDB IDs: 1HP7, 5IO1, 4PYW) obtained via molecular docking and dynamics simulations; (FIG. 8O) complexes formed by peptides KAWFKHWN-GSG (SEQ ID NO: 41), WHAKKSHF-GSG (SEQ ID NO: 45), KWKHSHKW-GSG (SEQ ID NO: 43), and WHAKKSKF-GSG (SEQ ID NO: 46) with glycosylated human AAT (PDB IDs: 7API, 8API, and 9API) obtained via molecular docking and dynamics simulations; and (FIG. 8P) complex of human AAT with two antibody Fab fragments (PDB IDs 6HX₄ and 613Z).

FIGS. 9A-9D: Breakthrough curves obtained by loading a solution of human AAT at 1 mg/mL in different buffers—namely, (FIG. 9A) 20 mM Bis-Tris HCl at 6.0, (FIG. 9B) PBS at 7.4, (FIG. 9C) 50 mM Tris HCl at 7.4, and (FIG. 9D) 50 mM Tris HCl at pH 8.0—onto AAT Select or peptide-Toyopearl resins (WHAKKSKFG (SEQ ID NO: 31), WHAKKSHFG (SEQ ID NO: 29)) at two values of residence time (RT: 2 and 5 min).

FIGS. 10A-10C: Poisson-Boltzman electrostatic surface (front and back) of AAT (PDB IDs: 7API, 8API, and 9API) (FIG. 10A) before and (FIG. 10B) after adsorption of Tris (yellow spheres) and peptides WHAKKSHF-GSG (green cartoon; SEQ ID NO: 45), KWKHSHKW-GSG (blue cartoon; SEQ ID NO: 43), and WHAKKSKF-GSG (red cartoon; SEQ ID NO: 46) at pH 7.0; (FIG. 10C) additional AAT:KWKHSHKWG pose (blue cartoon; SEQ ID NO: 29), note: a cluster of AAT:KWKHSHKWG (SEQ ID NO: 29) complexes with Cα RMSD<7.5 Å) formed at pH 6.0; and (FIG. 10D) AAT:KWKHSHKWG (SEQ ID NO: 29) interactions at pH 8.0.

FIG. 11: Chromatograms of purification of human AAT from a clarified CHO-S cell culture (AAT titer: 1.15 mg/mL; HCP titer: 0.5 mg/mL) using Alpha-1 antitrypsin Select, WHAKKSKFG-Toyopearl (SEQ ID NO: 31), and WHAKKSHFG-Toyopearl (SEQ ID NO: 30) resins. Labels: FT, flow-through; W, wash; E, elution; CIP, cleaning in place.

FIGS. 12A-12B: SEC analysis of the (FIG. 12A) flow-through and (FIG. 12B) wash fractions obtained from the chromatographic purification of human AAT from a clarified CHO-S cell culture (AAT titer: 1.15 mg/mL; HCP titer: 0.5 mg/mL) using AAT Select, WHAKKSHFG-Toyopearl (SEQ ID NO: 30), and WHAKKSKFG-Toyopearl (SEQ ID NO: 31) resins.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic amino acids (e.g., aspartate, glutamate), basic amino acids (e.g., lysine, arginine, histidine), non-polar amino acids (e.g., alanine, valine, leucine, isoleucine, proline, phenylalanine, methionine, tryptophan), uncharged polar amino acids amino acids amino acids (e.g., glycine, asparagine, glutamine, cysteine, serine, threonine, tyrosine), and aromatic amino acids (tryptophan, phenylalanine, tyrosine, histidine).

The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide Dis 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, a “mixture” comprises a target biologic of interest (for which purification is desired) and one or more contaminant or impurity. In some embodiments, the mixture is produced from a host cell or organism that expresses the protein of interest (either naturally or recombinantly). Such mixtures include, for example, cell cultures, cell lysates, and clarified bulk (e.g., clarified cell culture supernatant).

As described herein, the term “α-1 antitrypsin” (or AAT) refers to any naturally occurring or recombinantly or synthetically produced AAT from any species or organism (see, e.g., UniProt Ref. Q13747; and GenBank: CAA34982.1). In one embodiment, the AAT sequence of interest is identical to that disclosed in Long GL et al., Biochemistry 23(21):4828-4837, 1984. It represents the M allele, which is the most common and functional human α 1-antitrypsin; however, as would be recognized by one of ordinary skill in the art based on the present disclosure, the peptide ligand(s) described herein can potentially bind any mutant, variant, derivative, or fragment of AAT. In some cases, the leader sequence of the AAT sequence may be replaced with a different leader sequence, or the AAT may be engineered to include a tag or linker.

### 2. Compositions and Methods

α-1 antitrypsin (AAT) deficiency, a major risk factor for chronic obstructive pulmonary disease, is one of the most prevalent and fatal hereditary diseases. The rising demand of AAT poses a defined need for new processes of AAT manufacturing from recombinant sources. Commercial affinity adsorbents for AAT purification present the intrinsic limitations of protein ligands—chiefly, the high cost and the lability towards the proteases in the feedstocks and the cleaning-in-place utilized in biomanufacturing—which limit their application despite their high capacity and selectivity. The extraction of AAT from pooled plasma via precipitation-based fractionation and chromatographic purification has supported augmentation therapy to patients suffering from AATD for decades. The remarkable advancements in the expression of human proteins in engineered CHO cells, combined with the challenges of AAT isolation from plasma, suggest that the future of AAT biomanufacturing may turn towards the recombinant route. This poses an urgent need for affinity adsorbents that combine high binding capacity and selectivity with affordability and scalability.

In response, embodiments of the present disclosure provide novel affinity adsorbents that utilize peptide ligands to purify AAT from CHO cell culture harvests. The proposed adsorbents provide comparable product yield and purity, and lifetime compared to its commercial counterpart AAT Select resin, while surpassing it in terms of binding capacity and affordability. With an estimated cost of $6 K per liter (e.g., value is based on the costs of the peptide and the base resin as well as the cost of labor related to the peptide conjugation needed to produce 10 liters of peptide-functionalized adsorbent), the WHAKKSHFG-Toyopearl resin (SEQ ID NO: 30), for example, is far more cost-effective than AAT Select (Cytiva) and CaptureSelect™ AAT-XL Affinity Matrix (ThermoFisher).

As described further herein, an ensemble of ligand candidates identified via library screening were conjugated on Toyopearl resin and evaluated via experimental and in silico AAT-binding studies. Initial ranking based on equilibrium binding capacity indicated WHAKKSKFG-(12.9 mg of AAT per mL of resin; SEQ ID NO: 31), WHAKKSHFG- (16.3 mg/mL; SEQ ID NO: 30), and KWKHSHKWG- (15.8 mg/mL; SEQ ID NO: 29) Toyopearl resins as top performing adsorbents. Notably, the fitting of adsorption data to Langmuir isotherms concurred with molecular docking and dynamics in returning values of dissociation constant (K_{D}) between 1-10 μM. These peptide-based adsorbents were thus selected for AAT purification from CHO fluids, affording values of AAT binding capacity up to 13 grams per liter of resin, and product yield and purity up to 77% and 97%. WHAKKSHFG-Toyopearl (SEQ ID NO: 30) resin maintained its purification activity upon 20 consecutive uses, demonstrating its potential for AAT manufacturing from recombinant sources.

In accordance with the above, embodiments of the present disclosure include novel peptide ligands capable of binding α-1 antitrypsin (AAT), or fragments thereof, to facilitate their isolation and/or purification from processing fluid streams. In some embodiments, the peptide ligand(s) comprises at least 6 amino acids and (i) at least one histidine residue and/or at least one lysine residue; (ii) at least one asparagine residue, and/or at least one serine residue, and/or at least one glutamate residue; or (iii) at least one phenylalanine residue and/or at least one tryptophan residue; and (iv) the peptide ligand(s) binds α-1 antitrypsin. In some embodiments, the at least one peptide ligand further comprises an alanine residue.

In some embodiments, the peptide ligand(s) comprises at least one histidine residue and at least one lysine residue. In some embodiments, the peptide ligand(s) comprises at least one asparagine residue, at least one serine residue and at least one glutamate residue. In some embodiments, the peptide ligand(s) comprises at least one phenylalanine residue and at least one tryptophan residue. In some embodiments, the peptide ligand(s) comprises at least one histidine residue, at least one lysine residue, at least one asparagine residue, at least one serine residue, and at least one glutamate residue. In some embodiments, the peptide ligand(s) comprises at least one histidine residue, at least one lysine residue, at least one phenylalanine residue, and at least one tryptophan residue. In some embodiments, the peptide ligand(s) comprises at least one asparagine residue, at least one serine residue, at least one glutamate residue, at least one phenylalanine residue, and at least one tryptophan residue.

As would be recognized by one of ordinary skill in the art based on the present disclosure, amino acid residues can be categorized based on certain biophysical characteristic. For example, similar amino acids can be grouped into the families, e.g., acidic amino acids (e.g., aspartate, glutamate), basic amino acids (e.g., lysine, arginine, histidine), non-polar amino acids (e.g., alanine, valine, leucine, isoleucine, proline, phenylalanine, methionine, tryptophan), uncharged polar amino acids amino acids amino acids (e.g., glycine, asparagine, glutamine, cysteine, serine, threonine, tyrosine), and aromatic amino acids (tryptophan, phenylalanine, tyrosine, histidine). In some embodiments, the peptide ligands of the present disclosure comprise at least one histidine residue or at least one arginine residue. In some embodiments, the peptide ligands of the present disclosure comprise at least one histidine residue and at least one arginine residue. In some embodiments, the peptide ligand(s) of the present disclosure comprises one or more of alanine (A), glutamic acid (E), phenylalanine (F), histidine (H), isoleucine (I), asparagine (N), proline (P), arginine (R), threonine (T), and tryptophan (W).

In some embodiments, the peptide ligand(s) of the present disclosure comprise at least 6 amino acids. In some embodiments, the peptide ligand(s) of the present disclosure comprise at least 7 amino acids. In some embodiments, the peptide ligand(s) of the present disclosure comprise at least 8 amino acids. In some embodiments, the peptide ligand(s) of the present disclosure comprise at least 9 amino acids. In some embodiments, the peptide ligand(s) of the present disclosure comprise at least 10 amino acids. In some embodiments, the peptide ligand(s) of the present disclosure comprise at least 11 amino acids. In some embodiments, the peptide ligand(s) of the present disclosure comprise at least 12 amino acids. In some embodiments, the peptide ligand(s) of the present disclosure comprise at least 13 amino acids. In some embodiments, the peptide ligand(s) of the present disclosure comprise at least 14 amino acids. In some embodiments, the peptide ligand(s) of the present disclosure comprise at least 15 amino acids. In some embodiments, the peptide ligand(s) of the present disclosure comprise from about 6 to about 15 amino acids. In some embodiments, the peptide ligand(s) of the present disclosure comprise from about 6 to about 12 amino acids. In some embodiments, the peptide ligand(s) of the present disclosure comprise from about 6 to about 10 amino acids. In some embodiments, the peptide ligand(s) of the present disclosure comprise from about 6 to about 8 amino acids. In some embodiments, the peptide ligand(s) of the present disclosure comprise from about 9 to about 15 amino acids. In some embodiments, the peptide ligand(s) of the present disclosure comprise from about 12 to about 15 amino acids.

In some embodiments, the peptide ligand(s) binds α-1 antitrypsin (e.g., human AAT) at a binding site or epitope having certain characteristics. In some embodiments, the binding site has a pocket depth from about 12.5 Å to about 14.5 Å. In some embodiments, the binding site has a volume from about 1100 Å³ to about 1800 Å³. In some embodiments, the binding site has a pI from about 3.5 to about 5.5. In some embodiments, the binding site has a GRAVY index from about −0.05 to about −0.65. In some embodiments, the AAT binding site exhibits two of these characteristics. In some embodiments, the AAT binding site exhibits three of these characteristics. In some embodiments, the AAT binding site exhibits all four of these characteristics.

In some embodiments, the peptide ligand(s) exhibits a maximum equilibrium binding capacity (Qₘₐₓ) of at least about 3.0 mg of α-1 antitrypsin per mL resin. In some embodiments, the peptide ligand(s) exhibits a maximum equilibrium binding capacity (Qₘₐₓ) of at least about 4.0 mg of α-1 antitrypsin per mL resin. In some embodiments, the peptide ligand(s) exhibits a maximum equilibrium binding capacity (Qₘₐₓ) of at least about 5.0 mg of α-1 antitrypsin per mL resin. In some embodiments, the peptide ligand(s) exhibits a maximum equilibrium binding capacity (Qₘₐₓ) of at least about 6.0 mg of α-1 antitrypsin per mL resin. In some embodiments, the peptide ligand(s) exhibits a maximum equilibrium binding capacity (Qₘₐₓ) of at least about 7.0 mg of α-1 antitrypsin per mL resin. In some embodiments, the peptide ligand(s) exhibits a maximum equilibrium binding capacity (Qₘₐₓ) of at least about 8.0 mg of α-1 antitrypsin per mL resin. In some embodiments, the peptide ligand(s) exhibits a maximum equilibrium binding capacity (Qₘₐₓ) of at least about 9.0 mg of α-1 antitrypsin per mL resin. In some embodiments, the peptide ligand(s) exhibits a maximum equilibrium binding capacity (Qₘₐₓ) of at least about 10.0 mg of α-1 antitrypsin per mL resin. In some embodiments, the peptide ligand(s) exhibits a maximum equilibrium binding capacity (Qₘₐₓ) of at least about 11.0 mg of α-1 antitrypsin per mL resin. In some embodiments, the peptide ligand(s) exhibits a maximum equilibrium binding capacity (Qₘₐₓ) of at least about 12.0 mg of α-1 antitrypsin per mL resin. In some embodiments, the peptide ligand(s) exhibits a maximum equilibrium binding capacity (Qₘₐₓ) of at least about 13.0 mg of α-1 antitrypsin per mL resin. In some embodiments, the peptide ligand(s) exhibits a maximum equilibrium binding capacity (Qₘₐₓ) of at least about 14.0 mg of α-1 antitrypsin per mL resin. In some embodiments, the peptide ligand(s) exhibits a maximum equilibrium binding capacity (Qₘₐₓ) of at least about 15.0 mg of α-1 antitrypsin per mL resin. In some embodiments, the peptide ligand(s) exhibits a maximum equilibrium binding capacity (Qₘₐₓ) of at least about 16.0 mg of α-1 antitrypsin per mL resin.

In some embodiments, the peptide ligand(s) exhibits a disassociation constant (K_{D}) less than or equal to about 14.0×10⁻⁶ M. In some embodiments, the peptide ligand(s) exhibits a disassociation constant (K_{D}) less than or equal to about 13.0×10⁻⁶ M. In some embodiments, the peptide ligand(s) exhibits a disassociation constant (K_{D}) less than or equal to about 12.0×10⁻⁶ M. In some embodiments, the peptide ligand(s) exhibits a disassociation constant (K_{D}) less than or equal to about 11.0×10⁻⁶ M. In some embodiments, the peptide ligand(s) exhibits a disassociation constant (K_{D}) less than or equal to about 10.0×10⁻⁶ M. In some embodiments, the peptide ligand(s) exhibits a disassociation constant (K_{D}) less than or equal to about 9.0×10⁻⁶ M. In some embodiments, the peptide ligand(s) exhibits a disassociation constant (K_{D}) less than or equal to about 8.0×10⁻⁶ M. In some embodiments, the peptide ligand(s) exhibits a disassociation constant (K_{D}) less than or equal to about 7.0×10⁻⁶ M. In some embodiments, the peptide ligand(s) exhibits a disassociation constant (K_{D}) less than or equal to about 6.0×10⁻⁶ M. In some embodiments, the peptide ligand(s) exhibits a disassociation constant (K_{D}) less than or equal to about 5.0×10⁻⁶ M. In some embodiments, the peptide ligand(s) exhibits a disassociation constant (K_{D}) less than or equal to about 4.0×10⁻⁶ M. In some embodiments, the peptide ligand(s) exhibits a disassociation constant (K_{D}) less than or equal to about 3.0×10⁻⁶ M. In some embodiments, the peptide ligand(s) exhibits a disassociation constant (K_{D}) less than or equal to about 2.0×10⁻⁶ M. In some embodiments, the peptide ligand(s) exhibits a disassociation constant (K_{D}) less than or equal to about 1.0×10⁻⁶ M.

In some embodiments, the peptide ligand(s) exhibits a dynamic binding capacity (DBC₁₀%) from about 5 mg/mL to about 20 mg/mL. In some embodiments, the peptide ligand(s) exhibits a dynamic binding capacity (DBC₁₀%) from about 5 mg/mL to about 15 mg/mL. In some embodiments, the peptide ligand(s) exhibits a dynamic binding capacity (DBC₁₀%) from about 5 mg/mL to about 10 mg/mL. In some embodiments, the peptide ligand(s) exhibits a dynamic binding capacity (DBC₁₀%) from about 10 mg/mL to about 20 mg/mL. In some embodiments, the peptide ligand(s) exhibits a dynamic binding capacity (DBC₁₀%) from about 15 mg/mL to about 20 mg/mL.

In some embodiments, the peptide ligand(s) of the present disclosure comprises an amino acid sequence having at least 70% sequence identity with any of SEQ ID NOs: 1-23. In some embodiments, the peptide ligand(s) of the present disclosure comprises an amino acid sequence having at least 75% sequence identity with any of SEQ ID NOs: 1-23. In some embodiments, the peptide ligand(s) of the present disclosure comprises an amino acid sequence having at least 80% sequence identity with any of SEQ ID NOs: 1-23. In some embodiments, the peptide ligand(s) of the present disclosure comprises an amino acid sequence having at least 85% sequence identity with any of SEQ ID NOs: 1-23. In some embodiments, the peptide ligand(s) of the present disclosure comprises an amino acid sequence having at least 90% sequence identity with any of SEQ ID NOs: 1-23. In some embodiments, the peptide ligand(s) of the present disclosure comprises an amino acid sequence having at least 91% sequence identity with any of SEQ ID NOs: 1-23. In some embodiments, the peptide ligand(s) of the present disclosure comprises an amino acid sequence having at least 92% sequence identity with any of SEQ ID NOs: 1-23. In some embodiments, the peptide ligand(s) of the present disclosure comprises an amino acid sequence having at least 93% sequence identity with any of SEQ ID NOs: 1-23. In some embodiments, the peptide ligand(s) of the present disclosure comprises an amino acid sequence having at least 94% sequence identity with any of SEQ ID NOs: 1-23. In some embodiments, the peptide ligand(s) of the present disclosure comprises an amino acid sequence having at least 95% sequence identity with any of SEQ ID NOs: 1-23. In some embodiments, the peptide ligand(s) of the present disclosure comprises an amino acid sequence having at least 96% sequence identity with any of SEQ ID NOs: 1-23. In some embodiments, the peptide ligand(s) of the present disclosure comprises an amino acid sequence having at least 97% sequence identity with any of SEQ ID NOs: 1-23. In some embodiments, the peptide ligand(s) of the present disclosure comprises an amino acid sequence having at least 98% sequence identity with any of SEQ ID NOs: 1-23. In some embodiments, the peptide ligand(s) of the present disclosure comprises an amino acid sequence having at least 99% sequence identity with any of SEQ ID NOs: 1-23. In some embodiments, the peptide ligand(s) of the present disclosure comprises an amino acid sequence having 100% sequence identity with any of SEQ ID NOs: 1-23.

In some embodiments, the peptide ligand(s) of the present disclosure comprises an amino acid sequence having at least 70% sequence identity with any of SEQ ID NOs: 1-5. In some embodiments, the peptide ligand(s) of the present disclosure comprises an amino acid sequence having at least 75% sequence identity with any of SEQ ID NOs: 1-5. In some embodiments, the peptide ligand(s) of the present disclosure comprises an amino acid sequence having at least 80% sequence identity with any of SEQ ID NOs: 1-5. In some embodiments, the peptide ligand(s) of the present disclosure comprises an amino acid sequence having at least 85% sequence identity with any of SEQ ID NOs: 1-5. In some embodiments, the peptide ligand(s) of the present disclosure comprises an amino acid sequence having at least 90% sequence identity with any of SEQ ID NOs: 1-5. In some embodiments, the peptide ligand(s) of the present disclosure comprises an amino acid sequence having at least 91% sequence identity with any of SEQ ID NOs: 1-5. In some embodiments, the peptide ligand(s) of the present disclosure comprises an amino acid sequence having at least 92% sequence identity with any of SEQ ID NOs: 1-5. In some embodiments, the peptide ligand(s) of the present disclosure comprises an amino acid sequence having at least 93% sequence identity with any of SEQ ID NOs: 1-5. In some embodiments, the peptide ligand(s) of the present disclosure comprises an amino acid sequence having at least 94% sequence identity with any of SEQ ID NOs: 1-5. In some embodiments, the peptide ligand(s) of the present disclosure comprises an amino acid sequence having at least 95% sequence identity with any of SEQ ID NOs: 1-5. In some embodiments, the peptide ligand(s) of the present disclosure comprises an amino acid sequence having at least 96% sequence identity with any of SEQ ID NOs: 1-5. In some embodiments, the peptide ligand(s) of the present disclosure comprises an amino acid sequence having at least 97% sequence identity with any of SEQ ID NOs: 1-5. In some embodiments, the peptide ligand(s) of the present disclosure comprises an amino acid sequence having at least 98% sequence identity with any of SEQ ID NOs: 1-5. In some embodiments, the peptide ligand(s) of the present disclosure comprises an amino acid sequence having at least 99% sequence identity with any of SEQ ID NOs: 1-5. In some embodiments, the peptide ligand(s) of the present disclosure comprises an amino acid sequence having 100% sequence identity with any of SEQ ID NOs: 1-5.

In some embodiments, the peptide ligand(s) of the present disclosure comprises an amino acid sequence having at least 70% sequence identity with any of SEQ ID NOs: 8-23. In some embodiments, the peptide ligand(s) of the present disclosure comprises an amino acid sequence having at least 75% sequence identity with any of SEQ ID NOs: 8-23. In some embodiments, the peptide ligand(s) of the present disclosure comprises an amino acid sequence having at least 80% sequence identity with any of SEQ ID NOs: 8-23. In some embodiments, the peptide ligand(s) of the present disclosure comprises an amino acid sequence having at least 85% sequence identity with any of SEQ ID NOs: 8-23. In some embodiments, the peptide ligand(s) of the present disclosure comprises an amino acid sequence having at least 90% sequence identity with any of SEQ ID NOs: 8-23. In some embodiments, the peptide ligand(s) of the present disclosure comprises an amino acid sequence having at least 91% sequence identity with any of SEQ ID NOs: 8-23. In some embodiments, the peptide ligand(s) of the present disclosure comprises an amino acid sequence having at least 92% sequence identity with any of SEQ ID NOs: 8-23. In some embodiments, the peptide ligand(s) of the present disclosure comprises an amino acid sequence having at least 93% sequence identity with any of SEQ ID NOs: 8-23. In some embodiments, the peptide ligand(s) of the present disclosure comprises an amino acid sequence having at least 94% sequence identity with any of SEQ ID NOs: 8-23. In some embodiments, the peptide ligand(s) of the present disclosure comprises an amino acid sequence having at least 95% sequence identity with any of SEQ ID NOs: 8-23. In some embodiments, the peptide ligand(s) of the present disclosure comprises an amino acid sequence having at least 96% sequence identity with any of SEQ ID NOs: 8-23. In some embodiments, the peptide ligand(s) of the present disclosure comprises an amino acid sequence having at least 97% sequence identity with any of SEQ ID NOs: 8-23. In some embodiments, the peptide ligand(s) of the present disclosure comprises an amino acid sequence having at least 98% sequence identity with any of SEQ ID NOs: 8-23. In some embodiments, the peptide ligand(s) of the present disclosure comprises an amino acid sequence having at least 99% sequence identity with any of SEQ ID NOs: 8-23. In some embodiments, the peptide ligand(s) of the present disclosure comprises an amino acid sequence having 100% sequence identity with any of SEQ ID NOs: 8-23.

In some embodiments, the peptide ligand(s) of the present disclosure comprises an amino acid sequence having at least 70% sequence identity with SEQ ID NO: 11. In some embodiments, the peptide ligand(s) of the present disclosure comprises an amino acid sequence having at least 75% sequence identity with SEQ ID NO: 11. In some embodiments, the peptide ligand(s) of the present disclosure comprises an amino acid sequence having at least 80% sequence identity with SEQ ID NO: 11. In some embodiments, the peptide ligand(s) of the present disclosure comprises an amino acid sequence having at least 85% sequence identity with SEQ ID NO: 11. In some embodiments, the peptide ligand(s) of the present disclosure comprises an amino acid sequence having at least 90% sequence identity with SEQ ID NO: 11. In some embodiments, the peptide ligand(s) of the present disclosure comprises an amino acid sequence having at least 91% sequence identity with SEQ ID NO: 11. In some embodiments, the peptide ligand(s) of the present disclosure comprises an amino acid sequence having at least 92% sequence identity with SEQ ID NO: 11. In some embodiments, the peptide ligand(s) of the present disclosure comprises an amino acid sequence having at least 93% sequence identity with SEQ ID NO: 11. In some embodiments, the peptide ligand(s) of the present disclosure comprises an amino acid sequence having at least 94% sequence identity with SEQ ID NO: 11. In some embodiments, the peptide ligand(s) of the present disclosure comprises an amino acid sequence having at least 95% sequence identity with SEQ ID NO: 11. In some embodiments, the peptide ligand(s) of the present disclosure comprises an amino acid sequence having at least 96% sequence identity with SEQ ID NO: 11. In some embodiments, the peptide ligand(s) of the present disclosure comprises an amino acid sequence having at least 97% sequence identity with SEQ ID NO: 11. In some embodiments, the peptide ligand(s) of the present disclosure comprises an amino acid sequence having at least 98% sequence identity with SEQ ID NO: 11. In some embodiments, the peptide ligand(s) of the present disclosure comprises an amino acid sequence having at least 99% sequence identity with SEQ ID NO: 11. In some embodiments, the peptide ligand(s) of the present disclosure comprises an amino acid sequence having 100% sequence identity with SEQ ID NO: 11.

In some embodiments, the peptide ligand(s) of the present disclosure comprises an amino acid sequence having at least 70% sequence identity with SEQ ID NO: 14. In some embodiments, the peptide ligand(s) of the present disclosure comprises an amino acid sequence having at least 75% sequence identity with SEQ ID NO: 14. In some embodiments, the peptide ligand(s) of the present disclosure comprises an amino acid sequence having at least 80% sequence identity with SEQ ID NO: 14. In some embodiments, the peptide ligand(s) of the present disclosure comprises an amino acid sequence having at least 85% sequence identity with SEQ ID NO: 14. In some embodiments, the peptide ligand(s) of the present disclosure comprises an amino acid sequence having at least 90% sequence identity with SEQ ID NO: 14. In some embodiments, the peptide ligand(s) of the present disclosure comprises an amino acid sequence having at least 91% sequence identity with SEQ ID NO: 14. In some embodiments, the peptide ligand(s) of the present disclosure comprises an amino acid sequence having at least 92% sequence identity with SEQ ID NO: 14. In some embodiments, the peptide ligand(s) of the present disclosure comprises an amino acid sequence having at least 93% sequence identity with SEQ ID NO: 14. In some embodiments, the peptide ligand(s) of the present disclosure comprises an amino acid sequence having at least 94% sequence identity with SEQ ID NO: 14. In some embodiments, the peptide ligand(s) of the present disclosure comprises an amino acid sequence having at least 95% sequence identity with SEQ ID NO: 14. In some embodiments, the peptide ligand(s) of the present disclosure comprises an amino acid sequence having at least 96% sequence identity with SEQ ID NO: 14. In some embodiments, the peptide ligand(s) of the present disclosure comprises an amino acid sequence having at least 97% sequence identity with SEQ ID NO: 14. In some embodiments, the peptide ligand(s) of the present disclosure comprises an amino acid sequence having at least 98% sequence identity with SEQ ID NO: 14. In some embodiments, the peptide ligand(s) of the present disclosure comprises an amino acid sequence having at least 99% sequence identity with SEQ ID NO: 14. In some embodiments, the peptide ligand(s) of the present disclosure comprises an amino acid sequence having 100% sequence identity with SEQ ID NO: 14.

In some embodiments, the peptide ligand(s) of the present disclosure comprises an amino acid sequence having at least 70% sequence identity with SEQ ID NO: 15. In some embodiments, the peptide ligand(s) of the present disclosure comprises an amino acid sequence having at least 75% sequence identity with SEQ ID NO: 15. In some embodiments, the peptide ligand(s) of the present disclosure comprises an amino acid sequence having at least 80% sequence identity with SEQ ID NO: 15. In some embodiments, the peptide ligand(s) of the present disclosure comprises an amino acid sequence having at least 85% sequence identity with SEQ ID NO: 15. In some embodiments, the peptide ligand(s) of the present disclosure comprises an amino acid sequence having at least 90% sequence identity with SEQ ID NO: 15. In some embodiments, the peptide ligand(s) of the present disclosure comprises an amino acid sequence having at least 91% sequence identity with SEQ ID NO: 15. In some embodiments, the peptide ligand(s) of the present disclosure comprises an amino acid sequence having at least 92% sequence identity with SEQ ID NO: 15. In some embodiments, the peptide ligand(s) of the present disclosure comprises an amino acid sequence having at least 93% sequence identity with SEQ ID NO: 15. In some embodiments, the peptide ligand(s) of the present disclosure comprises an amino acid sequence having at least 94% sequence identity with SEQ ID NO: 15. In some embodiments, the peptide ligand(s) of the present disclosure comprises an amino acid sequence having at least 95% sequence identity with SEQ ID NO: 15. In some embodiments, the peptide ligand(s) of the present disclosure comprises an amino acid sequence having at least 96% sequence identity with SEQ ID NO: 15. In some embodiments, the peptide ligand(s) of the present disclosure comprises an amino acid sequence having at least 97% sequence identity with SEQ ID NO: 15. In some embodiments, the peptide ligand(s) of the present disclosure comprises an amino acid sequence having at least 98% sequence identity with SEQ ID NO: 15. In some embodiments, the peptide ligand(s) of the present disclosure comprises an amino acid sequence having at least 99% sequence identity with SEQ ID NO: 15. In some embodiments, the peptide ligand(s) of the present disclosure comprises an amino acid sequence having 100% sequence identity with SEQ ID NO: 15.

In some embodiments, the peptide ligand(s) of the present disclosure comprises a linker. In some embodiments, the linker is capable of binding to a solid support. In some embodiments, the linker is bound to the C-terminus of the peptide ligand, and wherein the linker comprises a Glyₙ or a [Gly-Ser-Gly]m, wherein 10≥n≥1 and 6≥m≥1. In some embodiments, the peptide ligand is bound to a solid support. In some embodiments, the solid support comprises a non-porous or porous particle, a membrane, a polymer surface, a fiber or a woven or non-woven fibermat, a hydrogel, a microplate, and/or a microfluidic device. In some embodiments, the porous particle is a chromatograph resin. In some embodiments, the solid support comprises polyacrylate, polyacrylamide, polyether, polyolefin, polyester, polysaccharide, iron oxide, silica, titania, and/or zirconia.

In accordance with the above, embodiments of the present disclosure include a composition for purifying α-1 antitrypsin (or a fragment thereof) from a fluid (e.g., a processing fluid stream) using any of the peptide ligands described herein. In some embodiments, the fluid is a cell culture fluid. In some embodiments, the fluid comprises a supernatant and/or a cellular lysate. In some embodiments, the fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof. In some embodiments, the biological fluid is derived from HEK293 cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the biological fluid is derived from yeast cells or fungal cells. In some embodiments, the yeast cells are Pichia pastoris cells.

In some embodiments, the fluid comprises a pH from about 3.0 to about 9.0. In some embodiments, the fluid comprises a pH from about 4.0 to about 9.0. In some embodiments, the fluid comprises a pH from about 5.0 to about 9.0. In some embodiments, the fluid comprises a pH from about 6.0 to about 9.0. In some embodiments, the fluid comprises a pH from about 7.0 to about 9.0. In some embodiments, the fluid comprises a pH from about 8.0 to about 9.0. In some embodiments, the fluid comprises a pH from about 3.0 to about 8.0. In some embodiments, the fluid comprises a pH from about 3.0 to about 7.0. In some embodiments, the fluid comprises a pH from about 3.0 to about 6.0. In some embodiments, the fluid comprises a pH from about 3.0 to about 5.0. In some embodiments, the fluid comprises a pH from about 3.0 to about 4.0. In some embodiments, the fluid comprises a pH from about 4.0 to about 8.0. In some embodiments, the fluid comprises a pH from about 5.0 to about 7.0.

Embodiments of the present disclosure also include an adsorbent for purifying α-1 antitrypsin (or a fragment thereof) from a fluid using any of the peptide ligands described herein. In some embodiments, an “adsorbent” is used herein generically to refer to the solid phase used in chromatography for which the mobile phase components exhibit a selective affinity. Because such affinity can take a variety of forms other than adsorption (including size exclusion or complexation), the term refers to solid phases that adsorb the components of a mixture and to solid phases that do not technically adsorb components from the mobile phase, but which nevertheless behave as an adsorbent by slowing the migration velocity of one component relative to another in a chromatographic system.

Embodiments of the present disclosure also include a method of purifying α-1 antitrypsin (or a fragment thereof) from a fluid. In accordance with these embodiments, the method includes contacting the fluid with any of the peptide ligands described herein, or any of the adsorbents described herein, under conditions sufficient for the peptide ligands or adsorbents to bind the α-1 antitrypsin; and eluting the α-1 antitrypsin from the peptide ligands. In some embodiments, “purified” when referring to a component or fraction indicates that its relative concentration (weight of component or fraction divided by the weight of all components or fractions in the mixture) is increased by at least 20%. In one series of embodiments, the relative concentration is increased by at least 40%, 50%, 60%, 75%, 100%, 150%, or 200%. A component or fraction can also be said to be purified when the relative concentration of components from which it is purified (weight of component or fraction from which it is purified divided by the weight of all components or fractions in the mixture) is decreased by at least 20%, 40%, 50%, 60%, 75%, 85%, 95%, 98% or 100%. In still another series of embodiments, the component or fraction is purified to a relative concentration of at least 50%, 65%, 75%, 85%, 90%, 97%, 98%, or 99%. When a component or fraction in one embodiment is “separated” from other components or fractions, it will be understood that in other embodiments the component or fraction is “purified” at levels provided herein.

In some embodiments, the conditions sufficient for the peptide ligand(s) to bind the α-1 antitrypsin comprise use of a buffer comprising 0.1-0.2 M NaCl in 10⁻³⁰ mM Bis-Tris HCl buffer at pH 5.5 to 6.5. In some embodiments, the conditions sufficient for the peptide ligand(s) to bind the α-1 antitrypsin comprise use of a buffer comprising 0.1-0.2 M NaCl in 40-60 mM Tris HCl buffer at pH 7.0 to pH 8.0. In some embodiments, the conditions sufficient for the peptide ligand(s) to bind the α-1 antitrypsin comprise use of a buffer comprising PBS buffer at pH 7.0 to pH 8.0. In some embodiments, eluting the α-1 antitrypsin from the peptide ligands comprises use of a buffer comprising 1-3 M MgCl₂ in PBS at pH 7.0 to 8.0.

In some embodiments, the methods described herein for purifying α-1 antitrypsin (or a fragment thereof) from a fluid result in a yield of at least 50%. In some embodiments, the methods described herein for purifying α-1 antitrypsin (or a fragment thereof) from a fluid result in a yield of at least 60%. In some embodiments, the methods described herein for purifying α-1 antitrypsin (or a fragment thereof) from a fluid result in a yield of at least 70%. In some embodiments, the methods described herein for purifying α-1 antitrypsin (or a fragment thereof) from a fluid result in a yield of at least 80%. In some embodiments, the methods described herein for purifying α-1 antitrypsin (or a fragment thereof) from a fluid result in a yield of at least 90%.

In some embodiments, the methods described here for purifying α-1 antitrypsin (or a fragment thereof) from a fluid result in a purity of at least 50%. In some embodiments, the methods described here for purifying α-1 antitrypsin (or a fragment thereof) from a fluid result in a purity of at least 60%. In some embodiments, the methods described here for purifying α-1 antitrypsin (or a fragment thereof) from a fluid result in a purity of at least 70%. In some embodiments, the methods described here for purifying α-1 antitrypsin (or a fragment thereof) from a fluid result in a purity of at least 80%. In some embodiments, the methods described here for purifying α-1 antitrypsin (or a fragment thereof) from a fluid result in a purity of at least 90%.

### 3. Materials and Methods

Materials. Aminomethyl ChemMatrix and HMBA-ChemMatrix (particle diameter: 75-150 μm, loading: 0.6 mmol per g resin) resins were sourced from PCAS Biomatrix, Inc. (Saint-Jean-sur-Richelieu, Quebec, Canada). The Toyopearl AF-Amino-650M resin was obtained from Tosoh Corporation (Tokyo, Japan). NHS-AlexaFluor 488 (AF488) and NHS-AlexaFluor 594 (NHS-AF594), N,N′-dimethylformamide (DMF), dichloromethane (DCM), methanol, and N-methyl-2-pyrrolidone (NMP) were obtained from Fisher Chemical (Hampton, NH, USA). Fluorenylmethoxycarbonyl- (Fmoc-) protected amino acids Fmoc-Gly-OH, Fmoc-Ser(tBu)-OH, Fmoc-Ile-OH, Fmoc-Ala-OH, Fmoc-Phe-OH, Fmoc-Tyr(tBu)-OH, Fmoc-Asp(OtBu)-OH, Fmoc-His(Trt)-OH, Fmoc-Arg(Pbf)-OH, Fmoc-Lys(Boc)-OH, Fmoc-Asn(Trt)-OH, and Fmoc-Glu(OtBu)-OH, Hexafluorophosphate Azabenzotriazole Tetramethyl Uronium (HATU), diisopropylethylamine (DIPEA), piperidine, and trifluoroacetic acid (TFA) were obtained from ChemImpex International (Wood Dale, IL, USA). Kaiser test kits, triisopropylsilane (TIPS), and 1,2-ethanedithiol (EDT) were obtained from Millipore Sigma (St. Louis, MO, USA).

Null CHO-S clarified cell culture harvest was provided by BTEC (Raleigh, NC, USA). Human Alpha-1 antitrypsin (AAT) in lyophilized form was a gift of CSL Behring (King of Prussia, PA, USA). Glacial acetic acid, hydrochloric acid, sodium acetate, glycine, sodium hydroxide, potassium chloride, sodium chloride (NaCl), magnesium chloride (MgCl₂), Tris HCl, Bis-Tris HCl, ethanol, and dithiothreitol (DTT) were purchased from Fisher Scientific (Hampton, NH, USA). Phosphate buffered saline (PBS) at pH 7.4 was purchased from MilliporeSigma (St. Louis, MO, USA). Vici Jour PEEK chromatography columns (2.1 mm ID, 30 mm length, 0.1 mL volume), Alltech chromatography columns (3.6 mm ID, 50 mm length, 0.5 mL volume), and 10 μm polyethylene frits were obtained from VWR International (Radnor, PA, USA). All chromatographic experiments were performed using a ÄKTA Pure system from Cytiva (Marlborough, MA, USA). The BioResolve SEC mAb Column, 200 Å, 2.5 μm, 7.8×300 mm, size exclusion chromatography column was obtained from Waters Inc. (Milford, MA, USA). The AAT Select column was from Cytiva (Marlborough, MA). The 10⁻²⁰% Tris-Glycine HCl SDS-PAGE gels and Coomassie blue stain were purchased from Bio-Rad Life Sciences (Carlsbad, CA, USA). A Pierce™ BCA Protein Assay Kit and SilverQuest™ Silver Staining Kit were purchased from Fisher Scientific™ (Pittsburgh, PA, USA).

Synthesis of peptide libraries on aminomethyl ChemMatrix resin and selected peptides on Toyopearl resin. Peptide synthesis was performed on a Syro I automated peptide synthesizer (Biotage, Uppsala, Sweden) using nine protected amino acids, namely Fmoc-Ala-OH, Fmoc-Asn(Trt)-OH, Fmoc-Glu-(OtBu)-OH, Fmoc-Phe-OH, Fmoc-His(Trt)-OH, Fmoc-Lys(Boc)-OH, Fmoc-Ile-OH, Fmoc-Trp(Boc)-OH, and Fmoc-Ser(tBu)-OH: each amino acid coupling step was performed at 45ºC for 20 min, using 3 equivalents (eq.) of protected amino acid at the concentration of 0.5 M, 3 eq. of HATU (0.5 M), and 6 eq. of DIPEA (0.5 M) in 5 mL of dry DMF. The yield of peptide conjugation was monitored after each amino acid via the Kaiser test. The removal of Fmoc protecting groups was performed at room temperature using 20% v/v piperidine in DMF. The 6-mer peptide library X₁-X₂-X₃-X₄-X₅-X₆ and 8-mer peptide library X₁-X₂-X₃-X₄-X₅-X₆-X₇-X₈ were synthesized on 2 g of HMBA-ChemMatrix resin preloaded with the tripeptide spacer GSG (G: glycine; S: serine) following the “split-couple-recombine” method. The selected peptides 8-mer peptides ANAKIKKK-GSG (SEQ ID NO: 38), FEKWAKAH-GSG (SEQ ID NO: 39), FSHHSWKF-GSG (SEQ ID NO: 40), KAWFKHWN-GSG (SEQ ID NO: 41), KHSKAIAA-GSG (SEQ ID NO: 42), KWKHSHKW-GSG (SEQ ID NO: 43), WHAKKSHF-GSG (SEQ ID NO: 45), WHAKKSKF-GSG (SEQ ID NO: 46), and the 6-mer peptides AAHFHK-GSG, KHAWIF-GSG (SEQ ID NO: 48), KFHAWN-GSG (SEQ ID NO: 47), NHNKIH-G (SEQ ID NO: 35), and SHWHWA-GSG (SEQ ID NO: 50) were synthesized on Toyopearl AF-Amino-650M resin at the density of ˜0.15 mmol of peptide per gram of resin. Following chain elongation, the peptides were deprotected via acidolysis using a cleavage cocktail containing TFA, thioanisole, anisole, and EDT (94/3/2/1) for 2 hrs. Following deprotection, the ChemMatrix library resins were rinsed with DCM and DMF and stored in DMF at 4ºC, whereas the peptide-Toyopearl resins were washed sequentially with DCM, DMF, methanol, and stored in 20% v/v aqueous methanol.

Fluorescent Labeling of AAT and CHO HCPs. The AAT and the host cell proteins (HCPs) contained in the CHO cell culture harvest were labeled using NHS-Alexafluor 594 (NHS-AF594, red) and NHS-Alexafluor 488 (NHS-AF488, green), respectively. Both dyes were initially dissolved in anhydrous DMSO to a concentration of 10 mg/mL. A volume of 1 μL of NHS-AF594 was slowly added to 100 μL of AAT solution at 2 mg/mL in PBS pH 7.4, while 200 μL of NHS-AF488 was added to 4 mL of null CHO-S cell culture fluid at 1.0 mg/mL total HCP concentration. The labeling reactions were allowed to proceed for 1 hr at room temperature, under dark and gentle agitation. The unreacted dyes were removed using 0.5 mL Zeba™ Dye and Biotin Removal Spin Columns (ThermoFisher Scientific, Waltham, MA). The concentration of the labeled proteins in solution was determined by Bradford assay. The absorbance of the solutions of AF488-labeled CHO HCPs and AF594-labeled AAT was measured by UV spectrophotometry at the wavelength of 490 and 590 nm, respectively, using a Synergy HI plate reader (Biotek, Winooski, VT).

Dual-Fluorescence Screening of Peptide Library against AAT in CHO cell culture fluid. A screening mix was initially prepared by spiking AF594-labeled AAT in AF488-labeled CHO cell culture fluid to obtain a final concentration of 1.0 mg/mL for AAT and 0.5 mg/mL for CHO host cell proteins. Aliquots of 10 μL of library beads were initially equilibrated with PBS at pH 7.4 and subsequently incubated with 40 μL of screening mix for 2 hrs at room temperature in dark. The beads were thoroughly washed with PBS at pH 7.4 and 0.1% v/v Tween 20 in PBS at pH 7.4 and sorted automatically using a microfluidic screening device. Beads displaying strong red-only fluorescence were selected, individually incubated with 100 μL of 0.1 M glycine buffer pH 2.5 for 1 hr at room temperature and in the dark to elute the bound AF594-labeled AAT, and rinsed with MilliQ water and acetonitrile. The beads were finally analyzed via Edman degradation using a PPSQ-33A protein sequence (Shimadzu, Kyoto, Japan) to sequence the selected peptides.

Static binding capacity and affinity of peptide-Toyopearl resins. The selected sequences were synthesized on Toyopearl AF-Amino 650 M resin as described herein and evaluated via isotherm binding studies of AAT in non-competitive mode (e.g., pure AAT in PBS at pH 7.4) as described in prior work. Aliquots of 50 μL of peptide-Toyopearl resin were transferred in a tube, swollen in 20% v/v aqueous methanol, washed in MilliQ water, and finally equilibrated in PBS at pH 7.4. The resin aliquots were individually incubated with 0.5 mL of a solution of AAT at different concentrations (0.1, 0.25, 0.5, 0.75, 1.0, 1.5, 2.0, and 5.0 mg/mL) for 1.5 hrs at room temperature under gentle rotation. After incubation, the resin aliquots were centrifuged and the supernatants collected as unbound (UB) fraction; the resins were also washed with 0.5 mL of PBS for 30 min, and the supernatants were combined with the corresponding UB fractions. These were finally analyzed by Micro BCA Protein Assay kit to determine the concentration C* of unbound AAT in solution at the equilibrium with the resin, from which the amount q of bound AAT per volume of resin was determined by mass balance. The values of q vs. C* were plotted and fit using a Langmuir isotherm (Equation 1) to determine the values of maximum binding capacity (Qₘₐₓ) and affinity dissociation constant (K_{D})).

$$q = \frac{{Q}_{max} \cdot {C}^{*}}{{K}_{D} + {C}^{*}} \qquad Equation 1$$

Dynamic binding capacity of peptide Toyopearl resins. The dynamic binding capacity at 10% breakthrough (DBC_{10%}, mg of AAT per mL resin) of the selected peptide-Toyopearl resins and the control AAT Select resin was measured. A volume of 0.1 mL of resin was wet packed in a Vici Jour PEEK column, washed with 10 column volumes (CVs) of 20% v/v ethanol, deionized water (3 CVs), and finally equilibrated with 10 CVs of binding buffer. The binding buffers used in this study were (i) 0.15M NaCl in 20 mM Bis-Tris HCl buffer at pH 6.0, (ii) PBS buffer at pH 7.4, and (iii and iv) 0.15M NaCl in 50 mM Tris HCl buffer at pH 7.4 or pH 8.0. A volume of 2 mL of solution of human AAT at 1 mg/mL in PBS buffer was continuously loaded on the column at the flow rate of either 0.05 mL/min (residence time, RT: 2 min) or 0.02 mL/min (RT: 5 min). After loading, the resin was washed with 10 CVs of binding buffer at the flow rate of 0.1 mL/min. AAT elution was then performed with 20 CVs of PBS in 2 M MgCl₂ at pH 7.4 at the flow rate of 0.2 mL/min. The resin was regenerated with 10 CVs of glycine buffer at pH 2.0 at the flow rate of 0.2 mL/min. The effluents were continuously monitored by UV spectrometry at 280 nm and the resulting chromatograms were utilized to calculate the DBC_{10%} of AAT.

Purification of AAT from CHO cell culture supernatant using peptide-Toyopearl resins. Each peptide-functionalized resin was individually wet packed in the 0.5 mL Alltech PEEK column, washed with 20% v/v ethanol (10 CVs) and deionized water (3 CVs), and equilibrated with 10 CVs of PBS at pH 7.4. A volume of 2.5 mL of AAT at ˜1 mg/mL in CHO-S cell culture supernatant (HCP titer˜0.35 mg/mL) was loaded on the column at the flow rate of 0.2 mL/min (RT: 2 min). After loading, the resin was washed with binding buffer (5 CVs) to recover the UV₂₈₀ nm baseline. AAT elution was then performed using 15 CVs of PBS solution with 2 M MgCl₂ at pH 7.4 at the flow rate of 0.5 mL/min. The resin was regenerated with 10 CVs of glycine buffer at pH 2.0 at the flow rate of 0.5 mL/min. The collected flow-through and elution fractions were analyzed by analytical HPLC to measure the AAT concentration and determine the values of product yield, and size exclusion chromatography (SEC-HPLC) and gel electrophoresis to determine the values of product purity.

Quantification of AAT yield. The AAT titer in the chromatographic fractions were measured via analytical affinity chromatography using an AAT Select column installed on a Waters Alliance 2690 separations module system with a Waters 2487 dual absorbance detector (Waters Corporation, Milford, MA, USA). The AAT Select resin was wet packed in a Vici Jour PEEK 2.1 mm ID×30 mm column (0.1 mL) and initially equilibrated with PBS buffer at pH 7.4. A volume of 50 μL of each sample or standard was loaded on the column and processed using the analytical method proceeded outlined in Table 1. The effluent was monitored by 280 nm absorbance (A280), and the AAT concentration was determined based on the area of the elution peak; pure AAT at 0.1, 0.2, 0.4, 0.6, 0.8, 1.0, and 2.0 mg/mL was utilized to construct the standard curve.

TABLE 1
HPLC method for AAT quantification via analytical
affinity chromatography using AAT Select resin.

| Time | Flowrate | PBS | 0.1M Glycine |
|---|---|---|---|
| (min) | (mL/min) | pH 7.4 | buffer, pH 2.5 |
| 0.00 | 0.5 | 100% | 0% |
| 2.00 | 0.5 | 100% | 0% |
| 2.01 | 0.5 | 0% | 100% |
| 6.00 | 0.5 | 0% | 100% |
| 6.01 | 0.5 | 100% | 0% |
| 10.00 | 0.5 | 100% | 0% |

The values of yield (Y, %) and binding capacity (Q, mg of AAT per mL resin) of AAT product were calculated using Equations 2 and 3, respectively.

$$Y = \frac{{C}_{AAT , El} \times {V}_{El}}{{C}_{AAT , L} \times {V}_{L}} \times 100 \% \qquad Equation 2$$

$$Q = \frac{{C}_{AAT , L} \times {V}_{L} - {C}_{AAT , FT} \times {V}_{FT}}{{V}_{R}} \qquad Equation 3$$

Wherein C_{AAT,FT}. C_{AAT,EI}, and C_{AAT,L} are the AAT concentration in the flow-through and elution fractions and the load, respectively; V_{FT}, V_{EI} and V_{L} are the volume of the flow-through and elution fractions and the load, respectively; and V_{R} is the volume of resin.

Quantification of AAT purity by size-exclusion chromatography (SEC). The collected fractions were also analyzed by analytical SEC using a BioResolve SEC mAb Column, 200 Å, 2.5 μm, 7.8×300 mm column (Waters, Milford, MA) operated with a 40-min isocratic method using PBS at pH 7.4 as mobile phase. A volume of 20 μL of sample was injected and the effluent continuously monitored by UV spectrometry at 280 nm absorbance (A280). The purity of AAT (P, %) was calculated using Equation 4.

$$P = \frac{{A}_{AAT , El}}{{A}_{AAT , El} + {A}_{HCP , El}} \times 100 \% \qquad Equation 4$$

Wherein A_{AAT,EI} and A_{HCP,EI} are the values of area of the peaks respectively related to AAT and CHO host cell proteins (based on the residence time of the peak) recorded in the SEC analysis of the elution fractions generated as described herein.

Quantification of AAT purity by sodium dodecyl sulfate polyacrylamide gel electrophoresis (SDS-PAGE). The collected fractions were analyzed by reducing SDS-PAGE using 4-20% Mini-PROTEAN™ TGX™ Precast protein gels (Bio-Rad, Hercules, CA) with Tris/Glycine/SDS buffer as running buffer. The fractions were diluted or concentrated to a total protein concentration of ˜0.5 mg/mL and a volume of 2 μL of different samples were loaded to the wells of SDS-PAGE gels. The sample stripes were concentrated under 80 V for about 30 min and separated under 120 V for about 1 hr. Then the gels were stained by a SilverQuest™ Silver Staining Kit (ThermoFisher, Waltham, MA). Finally, the stained protein stripes were imagined by the Gel Doc2000 imaging system (Bio-Rad).

In silico evaluation of AAT binding pockets and AAT:peptide complexes. The homology structure of human AAT was initially prepared using Protein Prep Wizard (PPW, Schrödinger, New York, NY) by correcting missing atoms and/or side chains (PRIME), removing salts and ligands, adding explicit hydrogens, and optimizing the hydrogen-bonding network on PDB IDs 1HP7, 5101, and 4PYW; the ionization state at pH 7.4 and minimization of the protein structure were finally performed using PROPKA. The adjusted structure was then analyzed using SiteMap to identify sites for ACP binding, and the sites with high S-score (>0.8) and D-score (>0.9) were selected for ACP docking.

Peptides ANAKIKKK-GSG (SEQ ID NO: 38), FEKWAKAH-GSG (SEQ ID NO: 39), FSHHSWKF-GSG (SEQ ID NO: 40), KAWFKHWN-GSG (SEQ ID NO: 41), KHSKAIAA-GSG (SEQ ID NO: 42), KWKHSHKW-GSG (SEQ ID NO: 43), WHAKKSHF-GSG (SEQ ID NO: 45), WHAKKSKF-GSG (SEQ ID NO: 46), AAHFHK-GSG (SEQ ID NO: 37), KHAWIF-GSG (SEQ ID NO: 48), KFHAWN-GSG (SEQ ID NO: 47), NHNKIH-G (SEQ ID NO: 35), and SHWHWA-GSG (SEQ ID NO: 50) were constructed using the molecular editor Avogadro. The equilibration and production steps were performed in AMBER using the Amber ff19SB force field. Briefly, every peptide was placed in a simulation box with periodic boundary and containing 1,000 water molecules (TIP3P model), and equilibrated with 10,000 steps of steepest gradient descent; the ACP was then heated to 300 K in an NVT ensemble for 250 ps with 1 fs time steps, and equilibrated to 1 atm with a 500-ps NPT simulation with 2 fs time steps. The production runs were performed in the NPT ensemble at T=300 K and P=1 atm using the Nose-Hoover thermostat and the Parrinello-Rahman barostat, respectively. The leap-frog algorithm was used to integrate the equations of motion, with integration steps of 2 fs, and the atomic coordinates were saved every 2 ps. All covalent bonds were constrained using the LINCS algorithm, the short-range electrostatic and Lennard-Jones interactions were calculated using cut-off values of 1.0 nm and 1.4 nm, and the particle-mesh Ewald method was utilized for long-range electrostatic interactions; the list of non-bonded interactions was updated every 5 fs using a cutoff of 1.4 nm.

Finally, the peptides were docked in silico against the putative binding sites on human AAT using the docking software HADDOCK (High Ambiguity Driven Protein-Protein Docking, v.2.4). The residues on the selected binding sites of AAT and the X₁-X_{6/8} residues on the peptides were marked as “active”, and the surrounding residues were marked as “passive.” The docked AAT: peptide structures were grouped in clusters of up to 20 complexes based on Cα RMSD and ranked using the dMM-PBSA score. Finally, the top AAT:peptide complexes were refined via 100-ns atomistic MD simulations and evaluated to estimate the free energy of binding (ΔG_{B}).

### 4. Examples

The accompanying Examples are offered as illustrative as a partial scope and particular embodiments of the disclosure and are not meant to be limiting of the scope of the disclosure.

Embodiments of the present disclosure include the discovery and development of AAT-binding peptides for use as affinity ligands in AAT purification. A focused library of peptides was initially designed; the amino acid compositions and sequence lengths of the peptides in this library were informed by the in-silico analysis of α-helices A-E of AAT. These form the core structure of AAT and, being removed from the reactive center loop (RCL), represents an ideal binding site for AAT capture. The peptide library was screened by implementing a dual-fluorescent selection method developed in prior work, which combines microfluidic technology and real time image analysis to accelerate the identification of candidate sequences and minimize the risk of false positives. A set of 15 candidate peptide ligands was conjugated onto polymethacrylate-based Toyopearl resins, and evaluated in terms of AAT dynamic binding capacity and purification from a Chinese hamster ovary (CHO) cell culture supernatant. Ligands WHAKKSKFG (SEQ ID NO: 31), WHAKKSHFG (SEQ ID NO: 30), and KAWFKHWNG (SEQ ID NO: 27) featured remarkable values of binding capacity, rivaling the control AAT Select resin. Adsorbent WHAKKSHFG-Toyopearl (SEQ ID NO: 30) resin afforded high values of AAT yield and purity, and maintained an excellent purification performance upon repeated usage, demonstrating its potential for AAT manufacturing from recombinant sources.

### Example 1

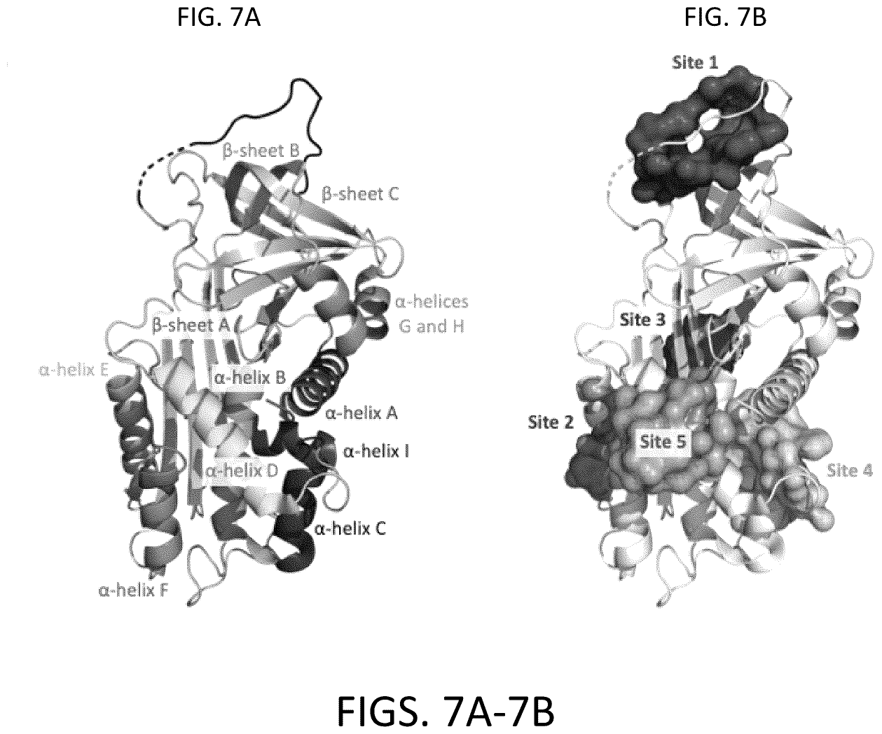

Tailoring a peptide library focused to the discovery of AAT-binding peptides. The core secondary structure of alpha-1 antitrypsin (AAT, ˜54 kDa, pI˜4.8) comprises nine α-helices (A-I) and three β-pleated sheets (A-C, FIG. 7A). The reactive center loop (RCL) of AAT, which bridges strand 5 of β-sheet A (s5A) to strand 5 of β-sheet C (s1C) and arches away from this core structure, features an inhibitory activity against neutrophil elastase, which protect tissues from enzymatic attack and inflammation. The inhibitory mechanism of AAT is initiated by the interaction between the RCL and the active site of neutrophil elastases. Depending on their sequence and orientation, these enzymes cleave dipeptide sequence P1-P1′ in the RCL region, resulting in a Michaelis-type complex between AAT and the elastase, which blocks the latter from triggering inflammatory effects. As an active site, the RCL and its surrounding domains (β-pleated sheets B and C, and α-helices G and H) are not suitable targets for peptide ligands, since the formation and dissociation of affinity complexes with ligands may cause structural and biochemical alterations, leading to unwanted loss of binding specificity and inhibitory activity towards target elastases. Notably, α-helices A-E of the core AAT structure are removed from the RCL region and represent an ideal target site for AAT capture.

An in silico “druggability” study was performed of the core AAT structure PDB IDs: 1HP7, 5IO1, 4PYW using SiteMap and identified 5 putative epitopes with appropriate morphological features—namely, pocket depth (PD), solvent-accessible surface area (SASA), and pocket volume (PV)) and biophysical properties (i.e., isoelectric point (pI), polarity, and grand average hydropathy (GRAVY) index) to harbor peptide ligands. The structure and properties of the identified epitopes are reported in FIG. 7B and Table 2, respectively.

TABLE 2
Structural and biophysical properties of target sites on human
AAT. Properties of the putative binding sites identified by analyzing
the crystal structure of human AAT (PDB IDs: 1HP7, 5IO1, 4PYW)
via SiteMap; the binding sites are labeled in FIG. 7.

| Binding | PD | SASA | PV | | | |
|---|---|---|---|---|---|---|
| site | (Å) | (Å²) | (Å³) | pI | Polarity | GRAVY |
| 1 | 12.9 | 155.2 | 1274.9 | 7.61 | 0.34 | −0.44 |
| 2 | 14.1 | 175.7 | 1629.8 | 5.21 | 0.49 | −0.65 |
| 3 | 12.7 | 144.0 | 1183.1 | 5.10 | 0.54 | −0.64 |
| 4 | 14.0 | 168.2 | 1599.8 | 5.16 | 0.48 | −0.16 |
| 5 | 12.7 | 150.0 | 1201.3 | 3.73 | 0.19 | −0.05 |

Four of the five epitopes are situated in the core structure of AAT and were therefore utilized to guide the design of a focused library of peptides; the fifth epitope overlapped with the reactive loop of AAT and was therefore excluded from targeted design. Notably, the four target binding sites feature a similar pocket depth (12.7-14.1 Å) and volume (1180-1600 Å³) as well as distinct negative electrostatic (pI˜3.7-5.2) and amphiphilic (GRAVY index˜−0.05 to −0.65) character. These properties recommend the use of small peptides (max. 10 amino acids) rich in cationic, polar, and aromatic residues. Accordingly, two libraries of linear peptides were constructed: (i) the peptide length was set at 6 and 8 residues, which balance the requirements of binding strength and selectivity with the considerations of cost and scalability; (ii) the combinatorial positions were randomized using amino acids capable of forming a network of non-covalent interactions with the target binding pockets, namely cationic residues histidine and lysine (H and K), polar residues asparagine, serine, and glutamate (N, S, and E), aromatic residues phenylalanine and tryptophan (F and W), and alanine (A) serving as a semi-rigid amino acid spacer; (iii) a GSG (Gly-Ser-Gly) tripeptide spacer was introduced between the combinatorial segment of the library and the resin to promote the display of the variable segment of the peptides, which improves the efficiency of both library screening and Edman sequencing. The peptide libraries were synthesized on ChemMatrix beads via Fmoc/tBu-based chemistry following the “split-couple-and-recombine” technique. ChemMatrix has been extensively utilized as a substrate for ligand selection: these porous, hydrophilic, translucent microparticles (pore diameter>100 nm, particle diameter˜200-250 μm) proved an excellent substrate for the synthesis and selection of candidate peptide ligands for chromatographic applications.

### Example 2

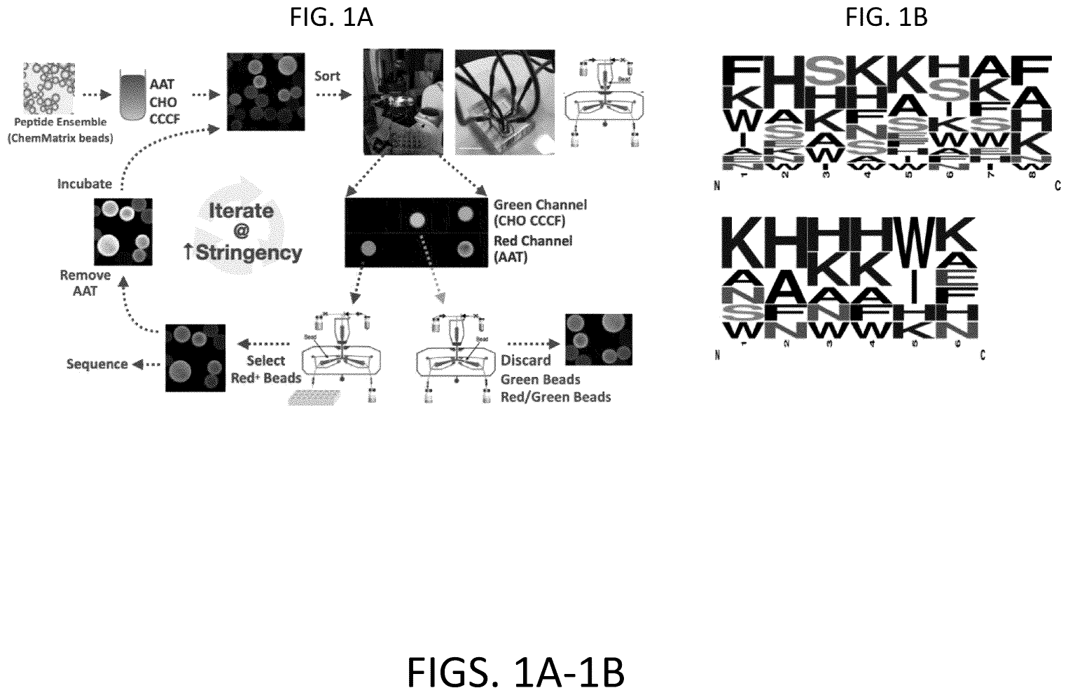

Combinatorial selection of peptide ligands against human AAT. A device developed previously for automating the rapid selection of peptide ligands targeting biological targets was used. The device sorts beads from solid-phase peptide libraries and provides (i) simultaneous positive and negative selection by dual fluorescent labeling to isolate peptides with high binding affinity and selectivity and (ii) rapid screening at a rate of ˜350 beads per hr. The device comprises a microfluidic bead-sorting chamber, a multiple wavelength fluorescent microscope, and software for real-time bead monitoring, image processing, and screening (FIG. 1A). The software performs rapid acquisition of fluorescent bead images and image analysis and instructs the sorting of a bead to either a selection receptacle or to waste. This bead-sorting device was used to screen the peptide library against a screening mix containing AAT labeled with red AlexaFluor 594 (AF594) spiked in a mixture of Chinese hamster ovary host cell proteins (CHO HCPs) collectively labeled with green AlexaFluor 488 (AF488). Library beads featuring a strong red-only fluorescence (high AAT-binding affinity and selectivity) were selected and analyzed by Edman degradation to identify the peptide sequences displayed thereon. The resultant ensemble of 13 candidate ligands (five 6-mer and eight 8-mer sequences) are listed in Table 3; the homology analysis of the sequences is reported in FIG. 1B.

TABLE 3
Sequences of 6-mer and 8-mer peptide ligands targeting human AAT.
The sequences were obtained from a library of peptide-ChemMatrix
beads screened against a screening feed comprising AF594-labeled
AAT and AF488-labeled CHO host cell proteins. The sequence homology
plot of the listed 8-mer and 6-mer peptides is reported in FIG. 1B.

| 6-mer sequences | 8-mer sequences | |
|---|---|---|
| AAHFHK | ANAKIKKK | AHIHFSEK |
| (SEQ ID NO: 1) | (SEQ ID NO: 8) | (SEQ ID NO: 16) |
| KFHAWN | FEKWAKAH | EHSSWHSK |
| (SEQ ID NO: 2) | (SEQ ID NO: 9) | (SEQ ID NO: 17) |
| KHAWIF | FSHHSWKF | IAKAKKWA |
| (SEQ ID NO: 3) | (SEQ ID NO: 10) | (SEQ ID NO: 18) |
| NHNKIH | KAWFKHWN | WNHSIFFH |
| (SEQ ID NO: 4) | (SEQ ID NO: 11) | (SEQ ID NO: 19) |
| SHWHWA | KHSKAIAA | NHSKKNIK |
| (SEQ ID NO: 5) | (SEQ ID NO: 12) | (SEQ ID NO: 20) |
| KAKHKE | KWKHSHKW | FKHNAISA |
| (SEQ ID NO: 6) | (SEQ ID NO: 13) | (SEQ ID NO: 21) |
| WNKKWK | WHAKKSHF | FHWNHWFH |
| (SEQ ID NO: 7) | (SEQ ID NO: 14) | (SEQ ID NO: 22) |
| | WHAKKSKF | ISSFEAAF |
| | (SEQ ID NO: 15) | (SEQ ID NO: 23) |

### Example 3

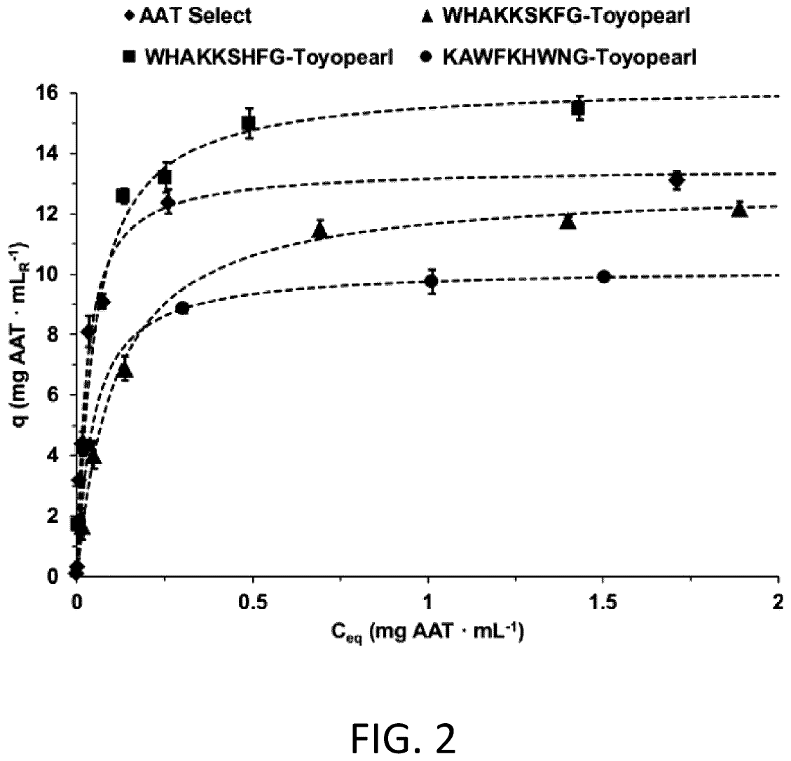

Evaluation of AAT-binding peptides via in vitro and in silico AAT binding studies. The selected peptides were synthesized on Toyopearl AF-Amino 650M resin via Fmoc/tBu synthesis and tested via static binding studies in non-competitive conditions (i.e., pure AAT in PBS at pH 7.4). Toyopearl resin was selected for ligand conjunction owing to its high chemical and mechanical stability as well as low non-specific protein adsorption, which make it an ideal substrate for direct peptide conjugation and protein chromatography. The AAT adsorption isotherms of ANAKIKKKG- (SEQ ID NO: 24), FEKWAKAHG- (SEQ ID NO: 25), FSHHSWKFG- (SEQ ID NO: 26), KAWFKHWNG- (SEQ ID NO: 27), KHSKAIAAG- (SEQ ID NO: 28), KWKHSHKWG- (SEQ ID NO: 29), WHAKKSHFG- (SEQ ID NO: 30), WHAKKSKFG- (SEQ ID NO: 31), AAHFHKG- (SEQ ID NO: 32), KHAWIFG- (SEQ ID NO: 33), KFHAWNG- (SEQ ID NO: 34), NHNKIHG- (SEQ ID NO: 35), and SHWHWAG-Toyopearl (SEQ ID NO: 36) resins were constructed to rank the candidate peptide ligands based on the values of maximum binding capacity (Qₘₐₓ˜mg of AAT per mL of resin) and affinity (i.e., dissociation constant, K_{D}˜μM) of the corresponding adsorbents. To this end, the adsorption data measured with the above-listed adsorbents across an ample range of AAT concentration (0.1-5 mg/mL) were fit against Langmuir isotherms (FIG. 2) and the resulting fitting parameters Qₘₐₓ and K_{D} are reported in Table 4. Notably, all adsorption plots fit the Langmuir isotherm well, returning values of K_{D} comprised within 0.35 and 6.9 UM, thus indicating that the peptides bind AAT via true affinity interaction. The values of Qₘₐₓ, however, fluctuated within a rather large interval, ranging from a minimum of 3.5 mg/mL, unacceptable for bioprocessing, to a maximum of 16.3 mg/mL, in line with that of AAT Select resin.

TABLE 4
Values of AAT maximum binding capacity (Qₘₐₓ) and dissociation constant
(K_{D}) of peptide-Toyopearl resins obtained by fitting the adsorption data in FIG. 2
using the Langmuir isotherm equation.

| | Qₘₐₓ | | | Qₘₐₓ | |
|---|---|---|---|---|---|
| | (mg_{AAT} per | | | (mg_{AAT} per | |
| Adsorbent | mL_{R}) | K_{D} (μM) | Adsorbent | mL_{R}) | K_{D} (μM) |
| ANAKIKKKG- | 3.51 | 6.70 | WHAKKSKFG- | 12.9 | 2.09 |
| Toyopearl | | | Toyopearl | | |
| (SEQ ID NO: 24) | | | (SEQ ID NO: 31) | | |
| FEKWAKAHG- | 5.10 | 4.67 | AAHFHKG-Toyopearl | 8.7 | 4.71 |
| Toyopearl | | | (SEQ ID NO: 32) | | |
| (SEQ ID NO: 25) | | | | | |
| FSHHSWKFG- | 9.4 | 3.63 | KHAWIFG-Toyopearl | 7.2 | 1.42 |
| Toyopearl | | | (SEQ ID NO: 33) | | |
| (SEQ ID NO: 26) | | | | | |
| KAWFKHWNG- | 10.2 | 0.87 | KFHAWNG-Toyopearl | 8.9 | 2.48 |
| Toyopearl | | | (SEQ ID NO: 34) | | |
| (SEQ ID NO: 27) | | | | | |
| KHSKAIAAG- | 9.1 | 3.62 | NHNKIHG-Toyopearl | 6.3 | 2.32 |
| Toyopearl | | | (SEQ ID NO: 35) | | |
| (SEQ ID NO: 28) | | | | | |
| KWKHSHKWG- | 15.8 | 0.63 | SHWHWAG- | 7.0 | 3.28 |
| Toyopearl | | | Toyopearl | | |
| (SEQ ID NO: 29) | | | (SEQ ID NO: 36) | | |
| WHAKKSHFG- | 16.3 | 0.95 | AAT Select | 13.5 | 0.51 |
| Toyopearl | | | | | |
| (SEQ ID NO: 30) | | | | | |

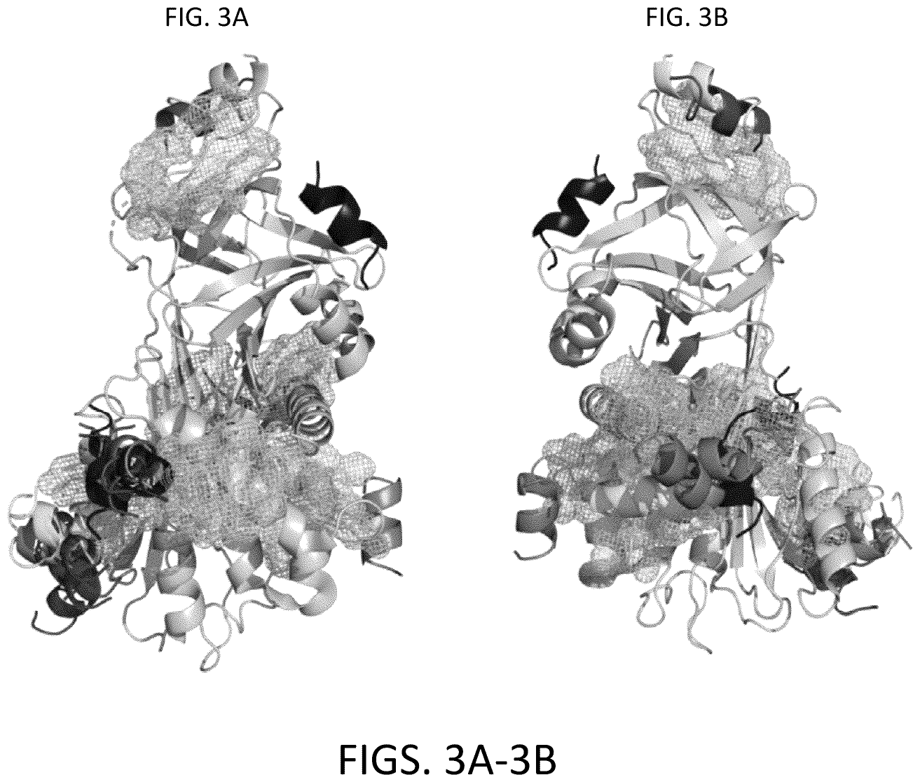

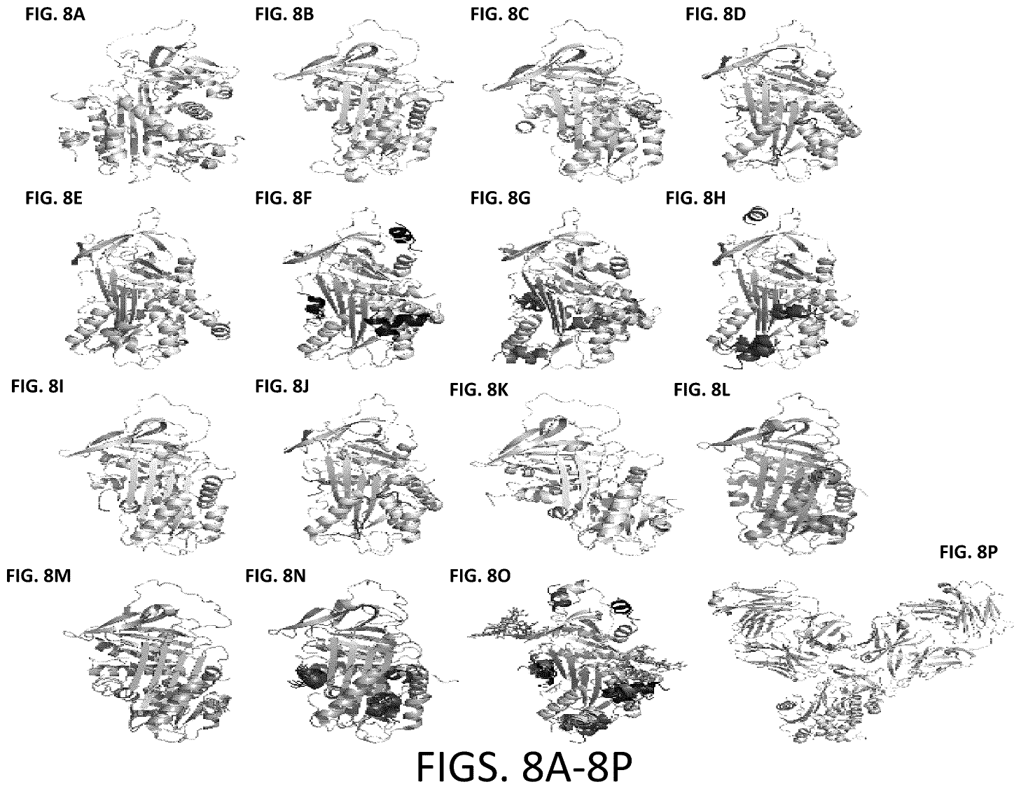

To gather a molecular-level understanding of the differences in binding strength among the various peptides, the sequences that featured the highest (WHAKKSKF-GSG (SEQ ID NO: 46), WHAKKSHF-GSG (SEQ ID NO: 45), KWKHSHKW-GSG (SEQ ID NO: 43), FSHHSWKF-GSG (SEQ ID NO: 40), and KHAWIF-GSG (SEQ ID NO: 48)) and lowest (AAHFHK-GSG (SEQ ID NO: 37), KHSKAIAA-GSG (SEQ ID NO: 42), FEKWAKAH-GSG (SEQ ID NO: 39), and ANAKIKKK-GSG (SEQ ID NO: 38)) binding performance were evaluated via molecular docking and dynamics analysis. The secondary structure of the peptides, obtained via MD simulations in explicit solvent, were docked against the homology structure of AAT obtained from published structures (PDB IDs: 1HP7, 5IO1, 4PYW). Docking was conducted in HADDOCK v. 2.4 and was focused on the putative binding sites obtained from an initial “druggability” study (FIG. 7B). Following a method developed in prior work, the −GSG tripeptide was appended on the C-terminal end of the peptides not to bind AAT: constraining the degrees of orientational freedom of the peptides mimics their conjugation onto the surface of the chromatographic resin, and has been consistently found to return values of K_{D,in silico} that match the corresponding values of K_{D,in vitro} more accurately than those obtained with full orientational freedom. The resulting complexes were refined via 100-ns MD simulations in explicit solvent to obtain values of binding free energy (ΔG_{b}) and calculate the corresponding K_{D,in silico}. The resulting complexes are reported for each peptide sequence in FIG. 8, while notable results are collated in FIG. 3; the resultant values of K_{D,in silico} are reported in Table 5 for each AAT site:peptide pair.

TABLE 5
Values of binding affinity (K_{D,in-silico}) of the complexes formed by selected
peptides with human AAT (PDB ID: 1HP7, 5IO1, 4PYW) obtained via molecular
docking and dynamics simulations. The binding affinity of the complexes
formed by AAT and antibodies were calculated by analyzing the PDB IDs
6HX4 and 613Z.

| Adsorbent | K_{D,in silico} (M) | Adsorbent | K_{D,in silico} (M) |
|---|---|---|---|
| AAHFHK-GSG | 8.2 · 10⁻⁶ | WFNHKSKG-GSG | 1.2 · 10⁻⁶ |
| (SEQ ID NO: 37) | | (SEQ ID NO: 44) | |
| ANAKIKKK-GSG | 2.3 · 10⁻⁵ | WHAKKSHF-GSG | 8.8 · 10⁻⁷ |
| (SEQ ID NO: 38) | | (SEQ ID NO: 45) | |
| FEKWAKAH-GSG | 7.1 · 10⁻⁶ | WHAKKSKF-GSG | 1.8 · 10⁻⁶ |
| (SEQ ID NO: 39) | | (SEQ ID NO: 46) | |
| FSHHSWKF-GSG | 6.1 · 10⁻⁶ | KFHAWN-GSG | 7.8 · 10⁻⁶ |
| (SEQ ID NO: 40) | | (SEQ ID NO: 47) | |
| KAWFKHWN-GSG | 3.0 · 10⁻⁶ | KHAWIF-GSG | 3.9 · 10⁻⁶ |
| (SEQ ID NO: 41) | | (SEQ ID NO: 48) | |
| KHSKAIAA-GSG | 9.3 · 10⁻⁷ | Fab1 | 7.9 · 10⁻⁶ |
| (SEQ ID NO: 42) | | (PDB ID: 6HX4) | |
| KWKHSHKW-GSG | 3.6 · 10⁻⁶ | Fab2 | 1.5 · 10⁻¹⁰ |
| (SEQ ID NO: 43) | | (PDB ID: 613Z) | |

Upon close examination of these results, four main conclusions were drawn. Firstly, the values of K_{D,in silico} align well with the values of K_{D,in vitro}, which is attributed to the directionality of peptide docking on AAT, thus confirming the ranking of the candidate ligands obtained from the binding isotherm studies; notably, the sequences featuring the lowest values of K_{D,in vitro} (highest affinity) were found to target neighboring binding sites on the AAT surface, suggesting that contiguous peptides on the surface of the resin may interact with one AAT molecule to form a multipoint interaction (a mechanism known as avidity). Secondly, most of the tested sequences indeed targeted the binding pockets localized in the core of AAT, indicating that the design of the library based on the initial druggability studies was conducted successfully; sequences KAWFKHWNG (SEQ ID NO: 27) and KWKHSHKWG (SEQ ID NO: 29), which bound the active loop, as well as ANAKIKKKG (SEQ ID NO: 24), FEKWAKAHG (SEQ ID NO: 25), and AAHFHKG (SEQ ID NO: 32), which bound with insufficient affinity, were discarded. Thirdly, the peptides with the highest number of putative binding sites on AAT—irrespective of their mutual position on the surface of the protein—afforded the highest binding capacity; this suggests that the ligands with more versatile AAT binding achieve a better protein coverage of the resin's surface, since proteins can be effectively adsorbed and retained in multiple spatial orientations, resulting in higher capacity. Fourthly, the putative binding sites of all the peptide ligands evaluated in this work are not involved in N-glycosylation (based on PDB IDs 7API, 8API, and 9API, FIG. 8); this result was corroborated by the AAT purification studies discussed below.

### Example 4

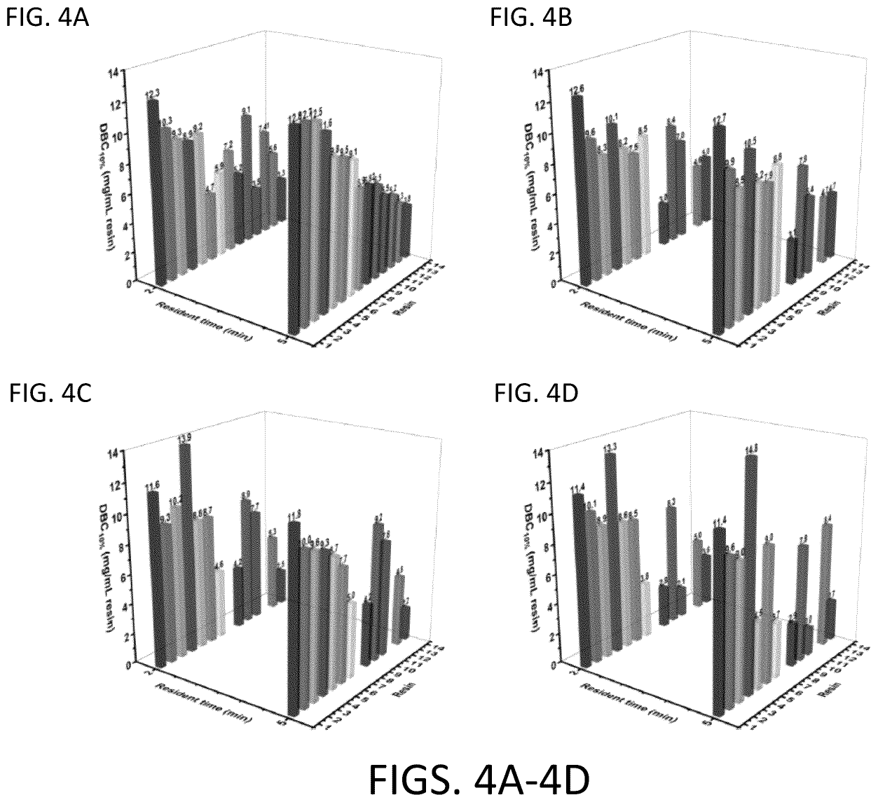

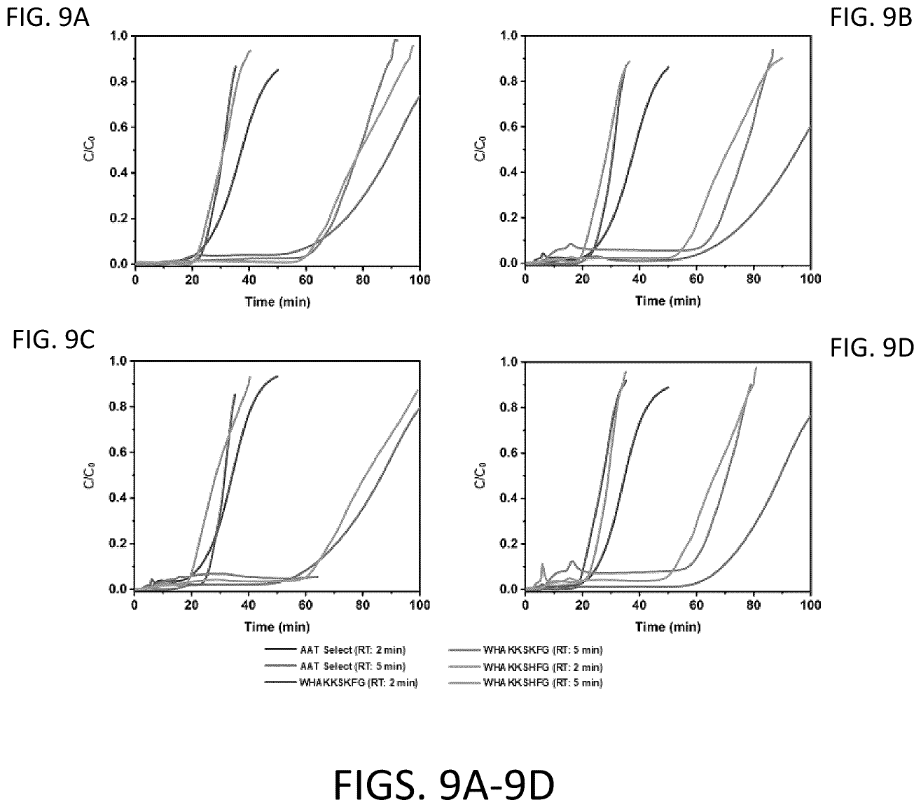

Evaluation of AAT-binding peptides via dynamic binding measurements. Following the equilibrium binding studies, the selected peptide-Toyopearl resins were evaluated under dynamic conditions to evaluate the interplay of kinetic factors—chiefly, the AAT transport within the chromatographic particles and the formation of AAT:peptide complexes—on the resultant binding capacity. To that end, the dynamic binding capacity at 10% breakthrough (DBC_{10%}, mg of AAT per mL of resin) of the peptide-Toyopearl resins was measured at two values of residence time (RT), namely 2 and 5 min, and four different buffer compositions, namely 20 mM Bis-Tris HCl buffer (0.15M NaCl) at pH 6.0, PBS buffer at pH 7.4, and 50 mM Tris HCl buffer (0.15M NaCl) at pH 7.4 and 8.0, and compared with that of AAT Select from Cytiva (FIG. 4; the corresponding breakthrough curves are reported in FIG. 9).

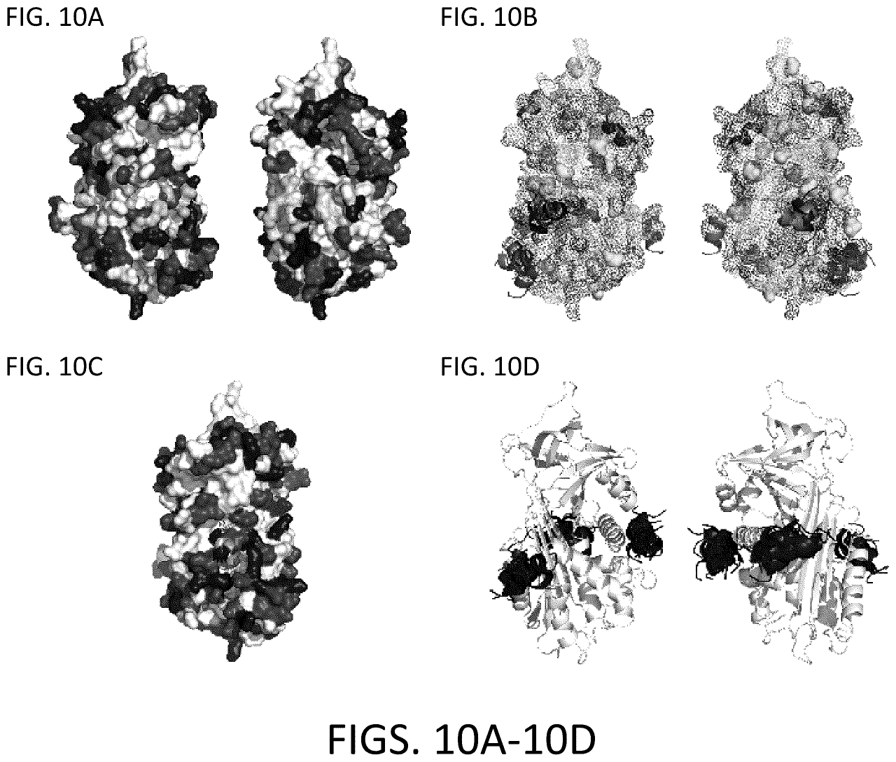

When measured in PBS at pH 7.4, the values of DBC_{10%} (RT of 5 min) of the top three peptide-Toyopearl resins were found to compare well with the control affinity adsorbent, AAT Select resin (12.8 mg/mL): specifically, WHAKKSKFG- (SEQ ID NO: 31), WHAKKSHFG-(SEQ ID NO: 30), and KWKHSHKWG (SEQ ID NO: 29)-Toyopearl resins featured 12.7 mg/mL, 12.5 mg/mL, and 11.6 mg/mL, respectively (FIG. 10A). Notably, these values of dynamic capacity and their equilibrium counterparts (Qₘₐₓ, Table 4) were nearly identical for AAT Select and WHAKKSKFG (SEQ ID NO: 31)-Toyopearl resins, while they differed for WHAKKSHFG (SEQ ID NO: 30)- and KWKHSHKWG (SEQ ID NO: 29)-Toyopearl resins. Furthermore, when the RT was lowered to 2 min, the DBC_{10%} of AAT Select resin decreased only slightly to 12.3 mg/mL, whereas WHAKKSKFG (SEQ ID NO: 31)-, WHAKKSHFG (SEQ ID NO: 30)-, and KWKHSHKWG (SEQ ID NO: 29)-Toyopearl resins presented a more substantial loss (˜20%) down to 10.3, 9.3, and 8.9 mg/mL, respectively. These results indicate that, as it is commonly observed, the mass transfer kinetics is a key determinant of binding capacity. Different variations in DBC_{10%} with feed flow rate are also expected given the different morphology of the beads: AAT Select is an agarose-based gel featuring an “open cell” porosity, whereas the peptide-Toyopearl adsorbents are polymethacrylate-based resin with channel-like pores; accordingly, the binding capacity of Toyopearl-based adsorbents may present a stronger dependence upon residence time (i.e., flowrate during adsorption) compared to AAT Select resin.

When measured in Tris buffer at comparable conductivity (˜15 mS/cm) and pH of 7.4, however, the values of DBC_{10%} did not show any appreciable variation with the feed flowrate: whether evaluated at the RT of 2 or 5 min, AAT Select maintained a binding capacity˜12 mg/mL, while WHAKKSKFG (SEQ ID NO: 31)- and WHAKKSHFG (SEQ ID NO: 30)-Toyopearl resins maintained values averaging at 9.7 and 9.3 mg/mL. The sole exception in this trend was represented by KWKHSHKWG (SEQ ID NO: 29)-Toyopearl resin, whose binding capacity in Tris buffer was significantly higher than its counterpart in PBS, especially at pH 6.0 and 8.0, reaching the highest value registered in this study (14.8 mg/mL).

To elucidate these phenomena, additional molecular docking and dynamics simulations were conducted of AAT:WHAKKSKFG (SEQ ID NO: 31), AAT: WHAKKSHFG (SEQ ID NO: 30), and AAT:KWKHSHKWG (SEQ ID NO: 29) binding in presence of Tris at pH 6.0, 7.0, and 8.0. At pH 7.0, Tris molecules adsorb onto the aspartate/glutamate-rich sites on the surface of AAT, thus mitigating its anionic character (the pI of AAT is 4.2-4.6). It was hypothesized that Tris complexation promotes AAT binding by minimizing the electrostatic repulsion between incoming AAT molecules and those already adsorbed on the pore surface (FIG. 10A). Furthermore, Tris molecules do not interfere with AAT:peptide interactions, because either they do not adsorb at the peptide-binding sites or adsorb weakly and are easily displaced by the peptide ligands (FIG. 10B).

Regarding ligand KWKHSHKWG (SEQ ID NO: 29), lowering the pH from 7.0 to 6.0 increases the positive charge of its two histidine (H) residues, resulting in a stronger interaction with AAT and an additional binding pose (i.e., a cluster of AAT:KWKHSHKWG (SEQ ID NO: 29) complexes with Cα RMSD<7.5 Å, FIG. 10C). Conversely, increasing the pH to 7.0 to 8.0 strengthens the contribution of the electrostatic interaction within the AAT: KWKHSHKWG (SEQ ID NO: 29) complexes: the anionic character of the AAT surface in correspondence of the putative binding sites increases with pH, thus promoting KWKHSHKWG (SEQ ID NO: 29) binding (FIG. 10D-10E).

Collectively, these in silico findings provide rationale to otherwise counterintuitive measurements of DBC₁₀% obtained at different values of residence time and properties of the mobile phase. By offering a valuable insight into the balance between the kinetics of mass transfer and AAT: peptide biorecognition, these results can be leveraged to optimize the chromatographic operation.

Having assigned a threshold of binding capacity at 10 mg/mL to ensure feasibility of the adsorbent in an AAT purification process, the adsorbents WHAKKSHFG (SEQ ID NO: 30)-, WHAKKSKFG (SEQ ID NO: 31)-, and KAWFKHWNG-Toyopearl (SEQ ID NO: 27) resins were selected for AAT purification from a CHO cell culture harvest.

### Example 5

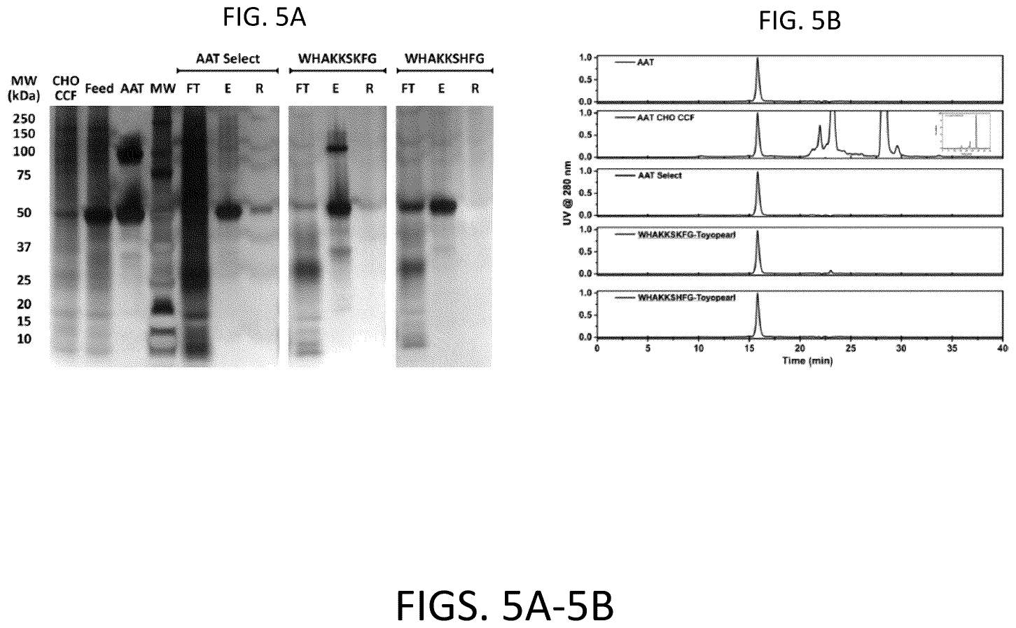

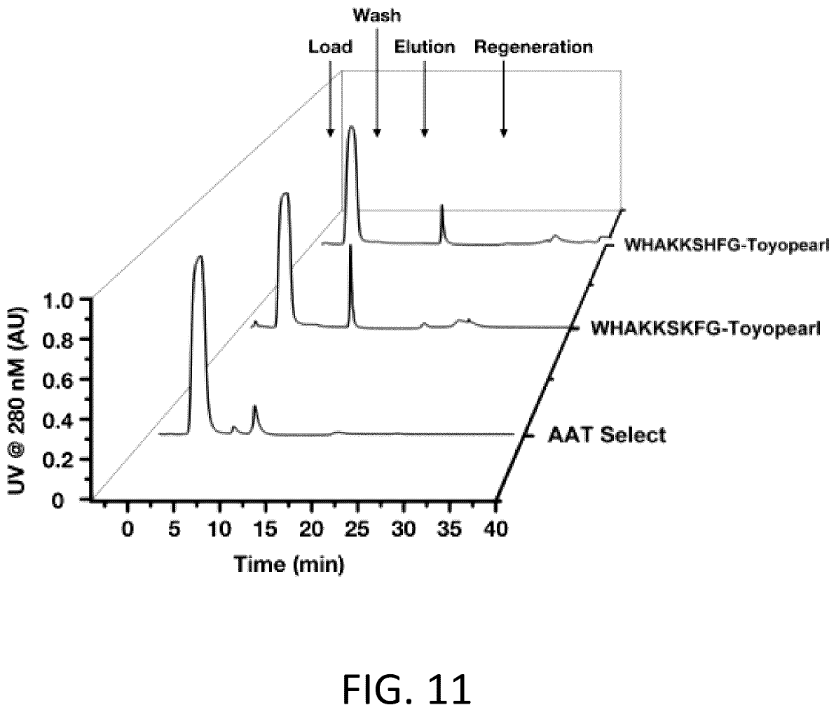

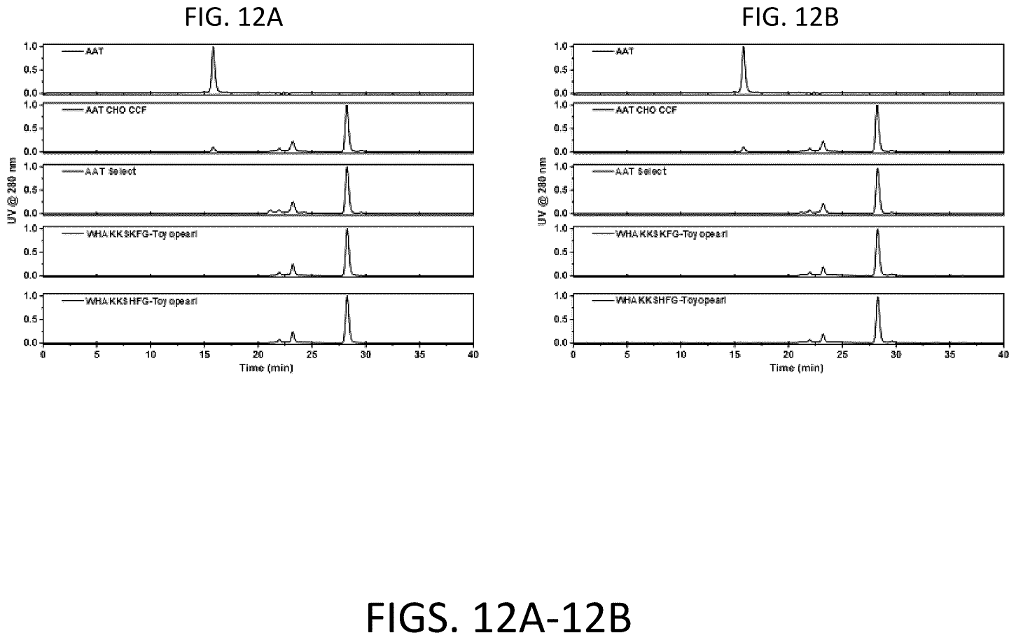

Purification of AAT from CHO cell culture harvests using AAT-binding adsorbents. Performing the library screening under competitive conditions, i.e., using a screening mix that combines AAT and CHO HCPs at a ratio that mimics industrial recombinant fluids, has been designed to afford candidate peptide ligands with selective biorecognition to ensure their ability to isolate AAT from complex sources in bind-and-elute mode. Adsorbents WHAKKSKFG (SEQ ID NO: 31)- and WHAKKSHFG (SEQ ID NO: 30)-Toyopearl resins, selected for their DBC₁₀%, were evaluated for the purification of human AAT from a clarified CHO-S cell culture (AAT titer: 1.15 mg/mL; HCP titer: 0.5 mg/mL). A relatively short RT of 2 min was adopted for the binding step to capitalize on the high AAT binding affinity and capacity of the peptide-based adsorbents while attempting to minimize the adsorption of CHO HCPs, as well as increase the productivity of the purification process. As with the control AAT Select resin, the bound AAT was eluted with 2M MgCl₂ in PBS at pH 7.4, and the regeneration of the resin was performed under acidic condition (pH 2.5). The chromatograms of AAT purification from the CHO-S CCF are compared in FIG. 11; the electrophoretic and SEC analyses of the collected fractions are reported in FIG. 5 and FIG. 12; and the resultant values of AAT yield and purity are collated in Table 6.

TABLE 6
Values of product yield and purity obtained by
purifying AAT from CHO cell culture fluids using
AAT Select and peptide-Toyopearl resins.

| Adsorbent | Yield | Purity |
|---|---|---|
| WHAKKSHFG-Toyopearl | 75.0% | 96.8% |
| (SEQ ID NO: 30) | | |
| WHAKKSKFG-Toyopearl | 76.6% | 93.1% |
| (SEQ ID NO: 31) | | |
| KAWFKHWNG-Toyopearl | 51.2% | 89.1% |
| (SEQ ID NO: 27) | | |
| AAT Select | 79.0% | 96.1% |

The electrophoretic analysis of the feedstock highlighted the presence of both monomeric AAT (˜54 kDa) and dimeric AAT (˜110 kDa; note: dimers resulting from AAT polymerization have been discussed in the literature); however, while the monomer is consistently found in all eluted fractions, the dimer is uniquely found in the fraction eluted from WHAKKSKFG (SEQ ID NO: 31)-Toyopearl resins. Conversely, the SEC chromatograms do not present any peak at ˜110 kDa, suggesting that the dimer is non-covalent in nature (and potentially an artifact of the electrophoresis). Despite the small discrepancies, the SDS-PAGE and SEC analysis of the eluted fractions concur in demonstrating that both peptide-based adsorbents bound AAT (i) selectively, with WHAKKSHFG (SEQ ID NO: 30)-Toyopearl resin affording a level of AAT purity (92.8%) comparable to that provided by AAT Select resin (97.1%); and (ii) with good capacity, since only minor amounts of AAT could be detected in the flow-through and wash fractions. All resins also eluted AAT efficiently and under mild conditions (2 M MgCl₂ in PBS at pH 7.4): WHAKKSKFG (SEQ ID NO: 31)- and WHAKKSHFG (SEQ ID NO: 30)-Toyopearl resins afforded values of AAT yield of 76.6% and 75.0%, respectively; AAT Select resin performed marginally better, with 79.0%. The AAT not released during elution with MgCl₂ was subsequently released during regeneration under acidic conditions. Conversely, KAWFKHWNG-Toyopearl (SEQ ID NO: 27) resin afforded a lower AAT purity (˜89.1%, data not shown) and insufficient yield (˜51.2%); the residual 27.6% of bound AAT was released during CIP.

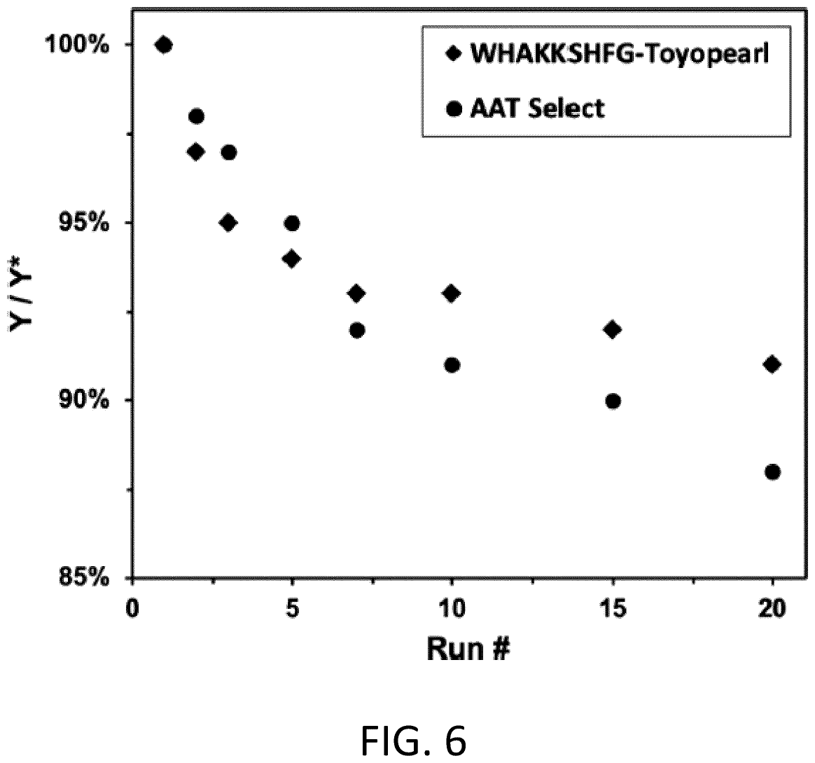

A life-time study was performed for WHAKKSHFG (SEQ ID NO: 30)-Toyopearl resin by conducting a series of 20 cycles of AAT purification from CHO CCF using aqueous 0.5 M sodium hydroxide for cleaning the resin following regeneration; caustic sanitization is in fact the industrial standard to ensure safety upon repeated usage of the resin. Notably, a rather minor decrease was observed in the binding capacity and AAT yield provided by WHAKKSHFG (SEQ ID NO: 30)-Toyopearl resins with increasing the number of cycles of purification, regeneration, and sanitization (FIG. 6); notably, the peptide-based adsorbent performed comparably to the control AAT Select resin. The exclusion of amino acids prone to alkaline-driven chemical degradation (i.e., asparagine, glutamine, cysteine, methionine) from the peptide sequence and the lack of a tertiary structure characteristic of short peptides make the selected ligand resistant to harsh chemicals, and ensure long lifetime and high reusability of the adsorbent.

1. A peptide ligand comprising at least 6 amino acids, wherein the peptide comprises:
(i) at least one histidine residue, and/or at least one lysine residue;
(ii) at least one asparagine residue, and/or at least one serine residue, and/or at least one glutamate residue; or
(iii) at least one phenylalanine residue, and/or at least one tryptophan residue; and
(iv) wherein the peptide ligand binds α-1 antitrypsin.

2. The peptide of claim 1, wherein the at least one peptide ligand further comprises an alanine residue.

3. The peptide ligand of claim 1, wherein the peptide ligand binds human α-1 antitrypsin.

4. The peptide ligand of claim 1, wherein the peptide ligand binds human α-1 antitrypsin at a binding site comprising at least one of the following characteristics:
(i) a pocket depth from about 12.5 Å to about 14.5 Å;
(ii) a volume from about 1100 Å³ to about 1800 Å³;
(iii) a pI from about 3.5 to about 5.5; and/or
(iv) a GRAVY index from about −0.05 to about −0.65.

5. The peptide ligand of claim 1, wherein the peptide ligand exhibits a maximum equilibrium binding capacity (Qₘₐₓ) of at least about 3.0 mg of α-1 antitrypsin per mL resin.

6. The peptide ligand of claim 1, wherein the peptide ligand exhibits a dynamic binding capacity (DBC_{10%}) from about 5 mg/mL to about 20 mg/mL.

7. The peptide ligand of claim 1, wherein the peptide ligand comprises an amino acid sequence having at least 90% sequence identity with any one of SEQ ID NOs: 1-23.

8. The peptide ligand of claim 1, wherein the peptide ligand comprises at least 6 amino acids and an amino acid sequence having at least 90% sequence identity with any one of SEQ ID NOs: 1-7.

9. The peptide ligand of claim 8, wherein:
(i) the peptide ligand comprises the amino acid sequence of any one of SEQ ID NOs: 1-5; or
(ii) the peptide ligand comprises the amino acid sequence of any one of SEQ ID NOs: 6 or 7.

10. (canceled)

11. The peptide ligand of claim 1, wherein the peptide ligand comprises at least 8 amino acids and an amino acid sequence having at least 90% sequence identity with any one of SEQ ID NOs: 8-23.

12. The peptide ligand of claim 11, wherein:
(i) the peptide ligand comprises the amino acid sequence of any one of SEQ ID NOs: 8-15; or
(ii) the peptide ligand comprises the amino acid sequence of any one of SEQ ID NOs: 16-23.

13. (canceled)

14. The peptide ligand of claim 1, wherein the peptide ligand comprises a linker, and wherein the linker is capable of binding to a solid support.

15. The peptide ligand of claim 14, wherein the linker is bound to the C-terminus of the peptide ligand, and wherein the linker comprises a Glyₙ or a [Gly-Ser-Gly]ₘ, wherein 10≥n≥1 and 6≥m≥1.

16. The peptide ligand of claim 1, wherein the peptide ligand is bound to a solid support, and wherein the solid support comprises a non-porous or porous particle, a membrane, a polymer surface, a fiber or a woven or non-woven fibermat, a hydrogel, a microplate, and/or a microfluidic device.

17-18. (canceled)

19. A composition for purifying α-1 antitrypsin, or a fragment thereof, from a fluid, wherein the composition comprises the peptide ligand of claim 1.

20. The composition of claim 19, wherein the fluid is a cell culture fluid.

21. (canceled)

22. The composition of claim 20, wherein the fluid is derived from CHO cells or HEK293 cells.

23-25. (canceled)

26. The composition of claim 19, wherein the fluid is derived from yeast cells or fungal cells.

27. (canceled)

28. An adsorbent for purifying α-1 antitrypsin from a fluid, wherein the adsorbent comprises the peptide ligand of claim 1.

29. A method of purifying α-1 antitrypsin, or a fragment thereof, from a fluid, the method comprising:
contacting the fluid with the peptide ligand of claim 1, under conditions sufficient for the peptide ligands to bind the α-1 antitrypsin; and
eluting the α-1 antitrypsin from the peptide ligands.

30-33. (canceled)
